(19) United States
(12) Patent Application Publication
(10) Pub. No.: US 2002/0164372 A1
(43) Pub. Date: Nov. 7, 2002

(54) Controlled release systems for polymers

(76) Inventor: Sidney Pestka, North Caldwell, NJ (US)

Correspondence Address: ROPES & GRAY, ONE INTERNATIONAL PLACE, BOSTON, MA, 02110-2624, US

(21) Appl. No.: 10/040,267

(22) Filed: Dec. 31, 2001

Related U.S. Application Data
(60) Provisional application No. 60/258,916, filed on Dec. 29, 2000.

(51) Int. Cl. A61K 9/26; A61K 38/17

(52) U.S. Cl. 424/469; 514/2

(57) ABSTRACT

The present invention relates to controlled release delivery of biologically active molecules from a solid composition prepared by exposure of the molecules to an organic compound. For instance, the organic compound is an organic solvent, such as an alcohol (e.g., preferably a lower alcohol, such as methanol, ethanol, isopropanol, n-propanol, n-butanol, isobutanol, t-butanol, etc.), a mixture of alcohols, an aldehyde, a ketone, a hydrocarbon (saturated or unsaturated), or an aromatic hydrocarbon. The solvent can be a mixture of different organic solvents, or the resulting formulation can be a mixture of, e.g., different lyophilized preparations, such as may be used to control the release profile of the resulting admixture.

### REFERENCE TO RELATED APPLICATIONS

This application claims priority to U.S. Provisional Patent Application No. 60/258,916 filed on Dec. 29, 2000, the specification of which is incorporated by reference herein.

### BACKGROUND OF THE INVENTION

With the advent of genetic engineering, the large-scale availability of many bioactive polymers, such as proteins, carbohydrates and nucleic acids, has been achieved. However, the administration of these recombinantly produced peptides and proteins presents a unique set of problems. In many cases the maintenance of the biological effect of these proteins requires long- term administration. Daily administration of these agents in aqueous vehicles is inconvenient and costly; sustained or prolonged release is preferred. In addition, proteins are highly unstable in an aqueous environment most suitable for administration.

Moreover, successful treatment of a variety of conditions is limited by the fact that agents known to effectively treat these conditions may have severe side effects, requiring low dosages to minimize these side effects. In other instances, the therapeutic agents may be very labile, or have very short half-lives requiring repeated administration. In still other instances, the long term administration of a pharmaceutical agent may be desired.

In all these cases, the ability to deliver a controlled dosage in a sustained fashion over a period of time may provide a solution.

### SUMMARY OF THE INVENTION

One aspect of the present invention relates to controlled release delivery of biologically active molecules from a solid composition prepared by exposure of the molecules to an organic compound. For instance, the organic compound is an organic solvent, such as an alcohol (e.g., preferably a lower alcohol, such as methanol, ethanol, isopropanol, n-propanol, n-butanol, isobutanol, t-butanol, etc.), a mixture of alcohols, an aldehyde, a ketone, a hydrocarbon (saturated or unsaturated), or an aromatic hydrocarbon. The solvent can be a mixture of different organic solvents, or the resulting formulation can be a mixture of, e.g., different lyophilized preparations, such as may be used to control the release profile of the resulting admixture.

The subject molecule to be formulated for controlled release can be an organic compounds. In certain embodiments, it is a polymer, preferably a biopolymer such as a protein, a peptide, a nucleic acid, an oligonucelotide, a carbohydrate, a ganglioside, or a glycan. The subject molecule can be a lipid, a sterol or other lipophilic moiety. The subject controlled delivery system can be used to deliver the controlled release of small molecules (e.g., organic compounds).

In certain embodiments, the subject preparations are prepared by precipitation and/or lyophilization.

### BRIEF DESCRIPTION OF THE DRAWINGS

FIGS. 1-5. Graphs showing various release profiles for BSA preparations.

FIGS. 6A-D. Effect salt concentration of formulation on release of HSA and IFN-α012. Solution I consisted of 9.0 mg of HSA (Immuno-U.S.) and 10 μg of IFN-α012 in 40% (w/w) n-propanol (0.364 g n-propanol) in H₂O for a total weight of 0.91 g. The various Solution II compositions consisted of various quantities of sodium acetate (1 M, pH 6.3) and deionized water and 0.040 g n-propanol to make solutions of 40% n-propanol and 250, 450, and 600 mM final sodium acetate concentrations with a total volume of 0.10 g. Solution II (0.10 g) was added to Solution I (0.91 g) with stirring to yield a final 1.01 g of each formulation. The final 1.01 g formulations containing 40% n-propanol and 25, 45, and 60 mM concentrations of sodium acetate were stirred in 2 ml glass vials for 6 hr at 24° C. and passed through 25G syringe needles just prior to separating supernatants from precipitates. The quantity of HSA and IFN-o012 in washed precipitates was determined as described in Materials and Methods. Release was performed in PBS/0.01% thimerosal. A & B. Absolute (mg) and percent release of precipitated HSA, respectively. C & D. Absolute (ng) and percent release of precipitated IFN-α012, respectively.

FIGS. 7A-B. Effect of cation species in formulation on release of HSA. Solution I consisted of 8.1 mg of HSA (Immuno-U.S.) in 40% (w/w) n-propanol in deionized water in a total volume of 0.91 ml. The various Solution II compositions consisted of adding none or 0.025 ml of various salt stocks (each at 1 M cation concentration, pH 6.3) to deionized water followed by n-propanol to make solutions 40% (w/w) n-propanol and 250 mM final cation concentration in a total volume of 0.10 ml. Solution 11 (0.10 ml) was added to 0.91 ml of Solution I with stirring to give a final 1.01 ml formulation having 40% (w/w) n-propanol. The final 1.01 ml formulations containing 40% n-propanol and no or 25 mM concentrations of potassium, sodium or magnesium acetate were stirred in 2 ml glass vials for 6 hr at 24° C. prior to separating supernatants from precipitates. The quantity of HSA in washed precipitates was determined as described in Materials and Methods. Release was performed in PBS/0.01% thimerosal. A & B. Absolute (mg) and percent release of precipitated HSA, respectively. Salts were sodium, potassium, and magnesium acetate (indicated by NaOAc, KOAc, and Mg(OAc)₂, respectively).

FIGS. 8A-B. Effect of cation species in formulation on release of IFN-α012. Solution I consisted of 45 mg of HSA (Immuno-U.S.) and 5.44 μg IFN-α012 in 40% (w/w) n-propanol in deionized water in a total volume of 4.55 ml. The various Solution II compositions consisted of adding 36 μl of 0.1 M acetic acid (to compensate for the buffer capacity of the HSA solution) and 0.250 g of potassium, sodium or magnesium acetate solution (each at pH 6.3) to 0.314 g of deionized water and 0.400 g of n-propanol to make solutions of 40% (w/w) n-propanol and 250 mM final acetate concentration in a total weight of 1 g. The potassium acetate solution was made with 0.980 g potassium acetate, 10.061 g water and 0.274 ml 1 M acetic acid. The sodium acetate solution was made with 0.823 g sodium acetate, 10.056 g water and 0.245 ml 1 M acetic 10 acid. The magnesium acetate solution was made with 2.144 g magnesium acetate, 10 g water and 0.200 ml 1 M acetic acid. Solution 11 (0.50 ml) was added to 4.55 ml of Solution I with stirring to give a final 5.05 ml formulation having 40% (w/w) n-propanol. The final formulations were stirred in 50 ml conical tubes for 6 hr at 24° C., the precipitates washed with 5 ml of PBS/0.01% thimerosal, then suspended in 5 ml PBS/0.01% thimerosal, then split into two individual 2.5 ml samples prior to separating supernatants from precipitates. Release data is from the precipitates from one 2.5 ml portion of the formulation. The amount of IFN-α012 in washed precipitates was determined as described in Materials and Methods. Release was performed in PBS/0.01% thimerosal. A & B. Absolute (ng) and percent release of precipitated IFN-α012, respectively. Salts were sodium, potassium, and magnesium acetate (indicated by 21 mM NaOAc, 20 mM KOAc, and 18 mM Mg(OAc)₂, respectively).

FIGS. 9A-B. Effect of aqueous solution pH of formulation on release of IFN-α012. Acetic acid (0.1 M) was used to adjust 5% HSA (Alpha Therapeutic) stock solutions to pH 5.0 or pH 7.0. Solution I consisted of 10 mg of HSA from either pH 5.0 or pH 7.0 HSA stock solutions, 6.83 μg IFN-aO12 and additional water to a total weight of 0.6 g. The final formulations were prepared by adding 0.4 g of n-propanol to Solution I with stirring to yield a concentration of 40% (w/w) n-propanol. Final 1 g formulations were stirred in 2 ml glass vials for 24 hr at 24° C. prior to separating supernatants from precipitates. The quantity of IFN-α012 in washed precipitates was determined as described in Materials and Methods. Release was performed in PBS/0.01% thimerosal. A & B. Absolute (ng) and percent release of precipitated IFN-α012, respectively.

FIGS. 10A-B. Effect of aqueous solution pH of formulation on release of HSA and IFN-α12. Solution I consisted of 45 mg of HSA (Immuno-U.S.) and 5.44 μg IFN-α12 in 40% (w/w) n-propanol in deionized water in a total volume of 4.55 ml. Solution II compositions were prepared as follows. Solution IIa: 1.55 ml of 1 M acetic acid was added to 0.82 g anhydrous sodium acetate and 10 g deionized water to adjust pH of this Solution A to 5.52; then 0.036 ml of 0.1 M acetic acid was added to 0.250 g of Solution A to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. Solution IIb: 0.40 ml of 1 M acetic acid was added to 0.82 g anhydrous sodium acetate and 10 g of deionized water to adjust pH of this Solution B to 6.13; then 0.036 ml of 0.1 M acetic acid was added to 0.250 g of Solution B to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. Solution IIc: 0.245 ml of 1 M acetic acid was added to 0.823 g anhydrous sodium acetate and 10.056 g deionized water to adjust pH of this Solution C to 6.31; then 0.036 ml of 0.1 M acetic acid was added to 0.250 g of Solution C to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. To prepare the final formulations, 0.50 ml from Solutions Ia, IIb, or IIc was added to 4.55 ml of Solution I with stirring to yield three 5.05 ml formulations having 40% (w/w) n-propanol and pH 5.52, pH 6.13 or pH 6.31, respectively. Final formulations were stirred in 50 ml conical tubes for 6 hr at 24° C., then split into two individual 2.52 ml samples prior to separating supernatants from precipitates. Release data is from one 2.52 ml portion of the formulation. The amount of IFN-α012 in washed precipitates was determined as described in Materials and Methods. Release was performed in PBS/0.01% thimerosal. A & B. Absolute (mg) and percent release of precipitated HSA, respectively. C & D. Absolute and percent release of precipitated IFN-α012, respectively.

FIGS. 11A-B. Effect of acid concentration of formulation on release of HSA and IFN-α001 from precipitates formed in the presence of 25 mM sodium acetate. Solution I consisted of 8.1 mg of HSA (Immuno-U.S.) and 0.92 μg IFN-α001 in 40% (w/w) n-propanol in deionized water in a total volume of 0.9 ml. Several Solution II formulations, Ia, IIb, IIc and IId, were prepared consisting of 0.004, 0.010, 0.015 and 0.025 ml of 0.1 M acetic acid, respectively, in 40% (w/w) n-propanol in deionized water. Solution III consisted of 1 M sodium acetate and 40% (w/w) n-propanol in deionized water in a total volume of 0.025 ml. Several Solution IV formulations, IVa, IVb, IVc and IVd, were prepared consisting of 0.071, 0.065, 0.060 and 0.050 ml of 40% (w/w) n-propanol, respectively, in deionized water. In preparing the final formulations, Solutions Ia, IIb, IIc and IId were matched with Solutions IVa, IVb, IVc and IVd, respectively. Solutions II, III and IV were mixed together then Solution I added rapidly to the mixture to give a final 1 ml formulation. This yielded a formulation having a final concentration of 25 mM sodium acetate, 40% (w/w) n-propanol and the final acetic acid concentrations indicated on the Figure. Formulations were stirred in 2 ml glass vials for 6 hr at 24° C. prior to separating supernatants from precipitates. After washing, precipitates were lyophilized 4 hr at <400 mTorr. The amount of HSA in washed precipitates was determined as described in Materials and Methods. Release was performed in PBS/0.01% thimerosal. A & B. Absolute (mg) and percent release of precipitated HSA, respectively. C & D. Absolute (ng) and percent release of precipitated IFN-α012, respectively.

FIGS. 12A-D. Effect of salt concentration of formulation on release of HSA and IFN-α001 from precipitates formed in the presence of 1.5 mM acetic acid. Solution I consisted of 8.1 mg of HSA (Immuno-U.S.) and 0.92 μg IFN-α001 in 40% (w/w) n-propanol in deionized water in a total volume of 0.9 ml. Solution II consisted of 0.1 M acetic acid and 40% (w/w) n-propanol in deionized water in a total volume of 0.015 ml. Several Solution III formulations, IIIa, IIIb, IIIc and IIId, were prepared consisting of 0, 0.015, 0.025 and 0.035 ml of 1 M sodium acetate, respectively, in 40% (w/w) n-propanol in deionized water. Several Solution IV formulations, IVa, IVb, IVc and IVd, were prepared consisting of 0.085, 0.070, 0.060 and 0.050 ml of 40% (wlw) n-propanol, respectively, in deionized water. In preparing the final formulations, Solutions IIIa, IIIb, IIIc and IIId were matched with Solutions IVa, IVb, IVc and IVd, respectively. Solutions II, III and IV were mixed together then Solution I added rapidly to the mixture to give a final 1 ml formulation. This yielded a final concentration of 1.5 mM acetic acid, 40% (w/w) n-propanol (w/w) and the final sodium concentrations indicated on the Figure. Formulations were stirred in 2 ml glass vials for 6 hr at 24° C. prior to separating supernatants from precipitates. After washing, precipitates were lyophilized 4 hr at <400 mTorr. The amounts of HSA and IFN-α001 in washed precipitates were determined as described in Materials and Methods. Release was performed in PBS/0.01% thimerosal. A & B. Absolute (mg) and percent release of precipitated HSA, respectively. C & D. Absolute (ng) and percent release of precipitated IFN-α001, respectively.

FIGS. 13A-B. Effect of salt concentration and pH of formulation on release of HSA with tertiary butanol precipitates. Acetic acid (0.1 M) was used to adjust 5% HSA stock solutions (Alpha Therapeutic) to pH 5.35 or 7.0. Solution I consisted of 18.0 mg of HSA from the pH 5.35 or pH 7.05% stock solution, 1.0 μg IFN-α012 and deionized water bringing the total solution weight to 0.375 g. To prepare Solutions Ia and IIb with NaCl concentrations of 0.02 M and 0.1 M, respectively, sufficient deionized water was added to 0.021 and 0.0043 ml of a 3.75 M NaCl solution to bring the total weight of each solution to 0.425 g. Both pH 5.35 and pH 7.0 variants of Solution I (0.375 g) were added to Solutions Ia and IIb to yield 0.80 g of the various combinations of pH and NaCl concentration as shown in the Figure prior to the addition of 0.31 or 0.47 g of tert-butyl alcohol to yield 28.1% and 36.9% (w/w) tert-butyl alcohol (see summary of the chart legends). Final 1.11-1.27 g formulations were stirred in 2 ml glass vials for 24 hr at 24° C. prior to separating supernatants from precipitates. The amount of HSA in washed precipitates was determined as described in Materials and Methods. Release was performed in PBS/0.01% thimerosal. A & B. Absolute (mg) and percent release of precipitated HSA, respectively.

FIG. 14. Effect of pH and salt concentration of formulation on threshold of precipitation of HSA by n-propanol. An 11% (w/w) HSA (USB) was dialyzed 3 times for 6 hr each time against 2 L deionized H₂O in a Pierce Slide_alyzer (15 ml capacity, No. 66410, lot #BJ44820B). The final concentration was analyzed by spectrophotometry at 280 nm to be 8.28% (w/w). This solution was diluted to 4% (w/w) with deionized water. Amounts (0.9 g) of 4% HSA were weighed into 2 ml glass vials. Sodium acetate (1 M), acetic acid (1 M), sodium hydroxide (1 M), and water were added in various combinations in a total weight of 0.1 g to yield the final sodium concentrations and pH values measured in 1 g formulations as shown in the Figure. Subsequently, n-propanol was added in about 50 μl increments with stirring, and the point at which initial precipitates were stable (did not re-dissolve with stirring within 5 minutes) was recorded. Connected data points indicate equivalent sodium concentrations at various pH and n-propanol (w/w) concentrations.

### DESCRIPTION OF THE INVENTION

I. Overview

The present invention relates to a controlled release delivery system and is based on the discovery that treatment of proteins and other molecules such as carbohydrates, nucleic acids, and other substances with organic compounds can modify their solubility in aqueous media. For example, in one embodiment the exposure of the proteins to the organic solvent (such as an alcohol) replaces the water molecules and other associated moieties with organic residues. In certain embodiments, the subject preparations are solids, e.g., powders or crystals formed by lyophilization, precipitation or the like.

The resulting preparations can provide prolonged release formulations of the proteins, e.g., suitable for sustained biological effects when used as pharmaceuticals or in other aqueous uses. The examples given refer to protein, but the principle can apply to other water soluble biopolymers as well such as peptides, carbohydrates, nucleic acids, oligonucleotides, lipids, glycans, gangliosides and other biopolymers. Small organic molecules and some inorganic molecules that are solvated with attached water residues can be treated in an analogous way to provide controlled delivery of the specific molecules.

Furthermore, solubility of proteins is also modulated by porttranslational modifications that can change the solubility of the proteins. The methods described can alter the solubility of the proteins with and without the post-translational modifications.

In certain embodiment, the biomolecules are precipitated from the aqueous solution by addition of organic solvents and then lyophilized. In alternative procedures, the solution can be lyophilized directly from solution containing organic solvents to provide for the dried material to be formulated into a controlled release system; the precipitated protein washed with aqueous solution and then formulated directly without lyophilization; or the dry protein treated with organic solvent, then formulated after removal of the solvent.

In certain preferred embodiments, the solvent is a an inert solvent, and even more preferably an anhydrous organic solvent. The solvent should not irreversibly denature the polymer, e.g., the timescale for renaturation, if any is requireed, should not be signiificantly longer than the rehydration process.

Formulation and size of the material can be controlled by the timing and method of precipitation and lyophilization conditions. Upon precipitation of the molecules, the precipitate is lyophilized to remove excess water and prevent water from immediately replacing the organic solvents. Colloidal suspensions without direct precipitation can be used to substitute for precipitation. The colloidal suspensions can be used to generate particles of small size. Furthermore, the mixtures can be lyophilized directly without precipitation or colloid formation to provide particles of different sizes dependent on the concentration of the molecules in the organic-aqueous media, the method of precipitation and the concentration of the protein solution. In some instances, inorganic molecules that can replace the water molecules on the molecules to be released slowly can be used in a total aqueous system to provide the same results. after lyophilization. The release is affected by the specific organic solvent used, the buffer used, and the particle size of the precipitated and/or lyophilized protein.

In addition, the method of invention permits greater tailoring of release profiles. The subject preparations can be made to exhibit short-term or long-term release kinetics, thereby providing either rapid or sustained release of macromolecules. In any event, the subject preparations have, relative to preparations of the polymer lyophilized from aqueous solutions, a reduced solubility in serum or other biological fluid, e.g., the solubility rate over a period of at least 24, 48, or even 168 hours (7 days) is at least 2 fold less than preparations of the polymer lyophilized from aqueous solution, and more preferably at least 10, 25, 50 or even 100 fold less.

In certain preferred embodiments, the subject compositions permit the release of biologically active compound at a rate which provides an average steady state dosage of at least the ED₅₀ for the active compound for a period of at least 2 days, and more preferably at least 7, 14, 21, 50, or even 100 days.

In certain preferred embodiments, the solvent(s) are chosen such that, when administered to a patient (particularly a human), the solvent released from the formulation is done so at a rate which remains below the IC₅₀ for deleterious side effects, if any, of the solvent, and more preferably at least 1, 2 or even 3 orders of magnitude below such IC₅₀ concentrations.

In certain embodiments, the organic agent is a polar protic solvent, such as for example, aliphatic alcohols, glycols, glycol ethers, and mixtures thereof. In certain preferred embodiments, the organic agent is a water-miscible polar protic solvent.

Biodegradable or non-biodegradable materials known in the art in the form of gels, microspheres, wafers or inplants can be mixed with the subject modified molecules.

These subject formulations can be used in parenteral, oral, intramuscular, subcutaneous, dermal, intravenous, intrarterial, intralesional, intrathecal or other sites of delivery for the treatment, prevention and diagnosis of many diseases.

Still another aspect of the invention relates to a method for doing business, e.g., for the preparation of pharmaceutical formulations for the treatment of humans or other animals. In an exemplary embodiment of such methods, there is provided a lyophilization facility for generating the lyophilized preparations described herein. The lyophilized preparations are packaged as e.g., pills, tablets, patches, injectables and the like, preferably at a government approved facility, e.g., an FDA-approved facility. In preferred embodiments, the lyophilized preparation is provided in single dosage form, even if packaged in larger lots.

II. Definitions

“Bioerodible” signifies that the material may be dissolved or digested into component molecules by the action of the environment or particularly by the action by living organisms, and optionally metabolized or digested into simpler constituents without poisoning or distressing the environment or the organism.

“Administered to a mammal” means that the composition containing an active ingredient is administered orally, parenterally, enterically, gastrically, topically, transdermally, subcutaneously, locally or systemically. The composition may optionally be administered together with a suitable pharmaceutical excipient, which may be a saline solution, ethyl cellulose, acetotephtalates, mannitol, lactose, starch, magnesium stearate, sodium saccharin, talcum, glucose, sucrose, carbonate, and the like.

“Sustained delivery” or “sustained time release” denotes that the active ingredient is released from the delivery vehicle at an ascertainable and manipulatable rate over a period of minutes, hours, days, weeks or months, ranging from about thirty minutes to about two months or longer.

Abbreviations

| | |
|---|---|
| HSA | Human serum albumin |
| HOAc | Acetic acid |
| NaOAc | Sodium acetate |
| KOAc | Potassium acetate |
| Mg(OAc)₂ | Magnesium acetate |
| IFN-α001 | Interferon α-001 |
| IFN-α012 | Interferon α-012 |
| PBS | Phosphate-buffered saline |

III. Exemplary Biopolymers

The biopolymers which may be used in the present invention include proteins, carbohydrates, nucleic acids and combinations thereof.

Advantageously, according to the present invention, the subject method can be used to formulate a protein which is pharmaceutically valuable or of value in the agri-foodstuffs industry. Proteins of interest include cytokines, growth factors, somatotropin, growth hormones, colony stimulating factors, , erythropoietin, plasminogen activators, enzymes, T-cell receptors, surface membrane proteins, lipoproteins, clotting factors, anticlotting factors, tumor necrosis factors, transport proteins, homing receptors, addressing, etc. Examples of mammalian polypeptides include molecules such as renin, a growth hormone, including human growth hormone; bovine growth hormone; growth hormone releasing factor; parathyroid hormone; thyroid stimulating hormone; lipoproteins; α-1-antitrypsin; insulin; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-α and -β; enkephalinase; RANTES (regulated on activation normally T-cell expressed and secreted); human macrophage inflammatory protein (MIP-1-α); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factors (TGF) such as TGF-α, TGF-β and BMPs; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins;

decay accelerating factor; antigens (e.g., bacterial and viral antigens); transport proteins; homing receptors; addressins; regulatory proteins; immunoglobulin-like proteins; antibodies; nucleases; and fragments of any of the above-listed polypeptides.

Other examples of suitable therapeutic and/or prophylactic biologically active agents include nucleic acids, such as antisense molecules; and small molecules, such as antibiotics, steroids, decongestants, neuroactive agents, anesthetics, sedatives, cardiovascular agents, anti-tumor agents, antineoplastics, antihistamines, hormones (e.g., thyroxine) and vitamins.

IV. Exemplary Methods

The rate of controlled release of the protein can be modified by many variables. The variables include rate of addition of organic solvent, time of protein (or other molecule) in organic solvent (time of exposure of protein to organic solvent), concentration of organic solvents for precipitation of the protein, concentration of the organic solvents prior to precipitation, concentration of the organic solvents prior to lyophilization from solution directly, organic and non-organic composition of media, temperature, concentration of cations, concentration of anions, rate of precipitation, pH, mixtures of organic solvents, stirring, agitation, presence of other proteins as carriers, presence of other proteins for controlled release of multiple proteins, protein stabilizers, dissolved gasses, reducing agents, oxidizing agents, mass to surface area of the particles, washing of samples prior to preparation for release, salt concentration, length of time exposed to modifier agents, concentration of the proteins or other polymer, inorganic compounds, type of organic compounds, for example. Inorganic cations can be monovalent, divalent, trivalent, tetravalent or pentavalent; inorganic anions can be monovalent, divalent, trivalent, tetravalent or pentavalent. In some embodients, lyophilization can be omitted. For example, the precipitate can be washed with a nonpolar solvent such as n-hexane to remove the organic solvent without affecting the protein; or the precipatate can be washed with an aqueous medium to remove the organic solvent removing the excess organic solvent from the protein mass. Furthermore, the precipitate can be washed and/or preincubated to remove soluble protein and eliminate the higher initial release rate.

Organic compound does not need to be solvent, just constituent in the mixture.

In addition, the protein precipitates can be placed into a variety of biodegradable or non- biodegradable materials known in the art in the form of gels, microspheres, wafers or implants. In these cases, the release is controlled by both the intrinsic protein release rate and the rate of release controlled by the gels, microspheres, wafers or implants. These formulations can be used in parenteral, oral, intramuscular, subcutaneous, dermal, intravenous, intrarterial, intralesional, intrathecal or other sites of delivery for the treatment, prevention and diagnosis of many diseases.

During equilibration of the protein with the solvent, the organic solvent used is attached to the protein in the precipitates. The organic solvent can be replaced partially or completely with other organic compounds soluble in the solution. The organic compounds can be active pharmaceuticals such as antibiotics, antimicrobial agents, aminoglycosides, chloramphenicol, macrolides, antifungals, cephalosporins, 3,4-dihydroxyphenylalanine (DOPA), adrenergic agonists, adrenergic antagonists, cholinergic agonists, cholinergic antagonists, muscarinic agonists, muscarinic antagonists, antiviral agents, sympathomimetics, sympatholytics, serotonin agonists, serotonin antagonists, antihypertensive agents, monoamine oxidase inhibitors, diuretics, antiarrhythmic drugs, phosphodiesterase inhibitors, digitalis glycosides, calcium antagonists, vasodilators, prostaglandins, autacoids, lipid lowering drugs, anticoagulants, fibrinolytics, platelet aggregation inhibitors, antidepressants, benzodiazepines, antiepileptics, antiparkinson agents, analgesics, opioids, opioid peptides, opiates, peptides, antiinflammatory drugs (NSAIDs, acetaminophen), barbiturates, peptide hormones, steroids, glucocorticoids, mineralocorticoids, estrogens, progestins, androgens, antiandrogens, thyroxine, triiodothyronine, cyclooxygenase inhibitors, growth hormone releasing hormone (GHRH), antineoplastic drugs, and antihistamines. The attached organic compounds (as drugs) linked to bovine or human serum albumin or other proteins such as immunoglobulins can then be delivered as the protein is released and dissolved. The proteins with attached organic solvents are thus able to be used as effective delivery systems. Furthermore, with the use of immunoglobulins and other proteins that can target to specific tissues or cells, the attached molecules can then be delivered to the tissues or cells.

Preparations made by the subject process can be either homogeneous or heterogeneous mixtures of active agents, or of preparations of active agents prepared under different conditions (e.g., using different solvents, etc).

The amount of a biologically active agent, which is contained in a specific preparation, is a therapeutically, prophylactically or diagnostically effective amount, which can be determined by a person of ordinary skill in the art taking into consideration factors such as body weight, condition to be treated, type of polymer used, and release rate from the preparation.

The biologically active agent can also be mixed with other excipients, such as stabilizers, surfactants, solubility agents and bulking agents. Stabilizers are added to maintain the potency of the agent over the duration of the agent's release. Suitable stabilizers include, for example, carbohydrates, amino acids, fatty acids and surfactants and are known to those skilled in the art. Solubility agents are added to modify the solubility of the agent in aqueous solution or, as the case may be, in organic solvents. Suitable solubility agents include complexing agents, such as albumin and protamine, which can be used to control the release rate of the agent. Bulking agents typically comprise inert materials.

In another embodiment, a biologically active agent can be lyophilized with a metal cation component, to further stabilize the agent and control the release rate of the biologically active agent.

The subject formulations, if used a therapeutics, may be administered to a human or animal by oral or parenteral administration, including intravenous, subcutaneous or intramuscular injection; administration by inhalation; intraarticular administration; mucosal administration; ophthalmic administration; and topical administration. Intravenous administration includes catheterization or angioplasty.

In other embodiments, the subject preparations can be used in non-therapeutic aqueous environments, such as for the release of agents (such as enzymes) into a water supply or water treatment facility.

In addition to the active agent, the formulation can include other suitable polymers, e.g., to permit the resulting formulation to be used to form a microparticle. In a preferred embodiment, a polymer used in this method is biocompatible. A polymer is biocompatible if the polymer, and any degradation products of the polymer, such as metabolic products, are non-toxic to humans or animals, to whom the polymer was administered, and also present no significant deleterious or untoward effects on the recipient's body, such as an immunological reaction at the injection site. Biocompatible polymers can be biodegradable polymers, non-biodegradable polymers, a blend thereof or copolymers thereof.

Suitable biocompatible, non-biodegradable polymers include, for instance, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof.

Suitable biocompatible, biodegradable polymers include, for example, poly(lactide)s, poly(glycolide)s, poly(lactide-co-glycolide)s, poly(lactic acid)s, poly(glycolic acid)s, polycarbonates, polyesteramides, polyanhydrides, poly(amino acids), polyorthoesters, polyacetals, polycyanoacrylates, polyetheresters, polycaprolactone, poly(dioxanone)s, poly(alkylene alkylate)s, polyurethanes, blends and copolymers thereof. Polymers comprising poly(lactides), copolymers of lactides and glycolides, blends thereof, or mixtures thereof are more preferred. Said polymers can be formed from monomers of a single isomeric type or a mixture of isomers.

A polymer used in this method can be blocked, unblocked or a blend of blocked and unblocked polymers. An unblocked polymer is as classically defined in the art, specifically having free carboxyl end groups. A blocked polymer is also as classically defined in the art, specifically having blocked carboxyl end groups. Generally, the blocking group is derived from the initiator of the polymerization reaction and is typically an alkyl radical.

In certain embodiments, the subject formulations are prepared by lyophilization. The simplest form of lyophilizer would consist of a vacuum chamber into which wet sample material could be placed, together with a means of removing water vapor so as to freeze the sample by evaporative cooling and freezing and then maintain the water-vapor pressure below the triple-point pressure.

### EXAMPLE 1

Release of bovine serum albumin (BSA) was measured up to 811 hours from samples of lyophilized protein precipitated from an alcohol/aqueous solution. This example briefly describes sample preparation and analytical methodology and presents results showing controlled release of BSA. The release is affected by the specific alcohol used, the buffer used, and the particle size of the precipitated and lyophilized protein.

Solutions of BSA (USB, Amersham Life Sciences, Cat. No. 10868) at 5% (w/w) were prepared in 0.01 M acetate buffer using an equivalent volume of 0.005 M sodium acetate and 0.005 M acetic acid. The pH was approximately 5. The alcohol n-propanol was added to a concentration of 40% (v/v). After overnight equilibration at room temperature, the supernatant was removed and the precipitate frozen at −20 C and brought to −70 C before lyophilization. The surface upon which the vials were placed and the lyophilizer chamber was precooled to maintain the samples frozen during the lyophilization procedure. The sample was lyophilized for 5 hours. The time of lyophilization can be longer or shorter depending on the volume to be lyophilized. The lyophilized sample was divided into several pieces with a spatula. The pieces were divided into small particles by crushing the pieces against the wall and bottom of the glass vial. The larger masses and small crushed particles were weighed so that 5 to 10 mg of the masses and the crushed particles were placed into separate 1.5 ml conical polypropylene tubes, then 1 ml of phosphate buffered saline was added. The masses or particles were disbursed into the liquid. One hour after disbursing the samples, the contents of the tubes were mixed again and then the tubes centrifuged for 5 minutes at 5,000 rpm (Eppendorf Centrifuge, Model No. 5415). A sample of 0.1 ml was removed for assay and replaced with 0.1 ml of PBS. This procedure was repeated to take samples at 65 hours. At 98 hours and each time point thereafter, the full volume of release medium was removed and replaced with a fresh 1 ml of PBS.

Samples were analyzed for protein content with the microassay procedure for microtiter plates (Bio-Rad protein assay, based on the method of Bradford; Coomassie Brilliant Blue Dye, Cat. No. 500-0006) with 96 well microtiter plates. Standards contained 5 to 60 μg/ml of BSA. Standards and samples were added to the wells in a volume of 0.16 ml first, then 40 μl of dye was added to each well with mixing before reading the absorbance at 630 nm. Standard curves were constructed from absorbances corrected for the blank values in the absence of added protein (BSA). Protein concentrations of the samples were calculated from the standard curve that was based on the same lot of BSA and prepared on the basis of weight of BSA to total volume (w/v). The values for the protein released at various times were adjusted by determining differences in the protein concentration of the lyophilized BSA that was weighed and placed in solution from the BSA taken directly from the bottle of the commercial supplier (USB, Amersham Life Sciences, Cat. No. 10868) and placed in solution.

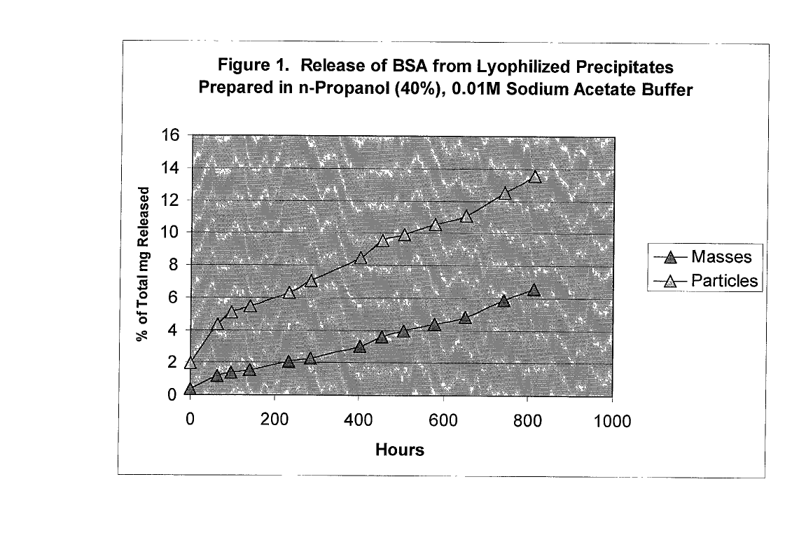

The results of the controlled release are shown in FIG. 1 [nP, represents n-propanol]. As can be seen, there is little or no burst effect and the release is essentially linear. The smaller particles with a large surface area to mass ratio release at a faster rate. There appears to be a slightly faster rate of release during the first hours of release (FIG. 1). This faster release rate can be eliminated by preincubating the samples in medium prior to use.

### EXAMPLE 2

Release of BSA was measured up to 811 hours from samples of lyophilized protein precipitated from alcohol/aqueous solution. This example briefly describes sample preparation and analytical methodology and presents results showing controlled release of BSA. The release is affected by the specific alcohol used, the buffer used, and the particle size of the precipitated and lyophilized protein.

Solutions of BSA (USB, Amersham Life Sciences, Cat. No. 10868) at 5% (w/w) were prepared in 0.1 M acetate buffer using an equivalent volume of 0.05 M sodium acetate and 0.05 M acetic acid. The pH was approximately 5. The alcohol n-propanol was added to a concentration of 50% (v/v). After overnight equilibration at room temperature, the supernatant was removed and the precipitate frozen at −20 C and brought to −70 C before lyophilization. The surface upon which the vials were placed and the lyophilizer chamber was precooled to maintain the samples frozen during the lyophilization procedure. The sample was lyophilized for 5 hours. The time of lyophilization can be longer or shorter depending on the volume to be lyophilized. The lyophilized sample was divided into several pieces with a spatula. The pieces were divided into small particles by crushing the pieces against the wall and bottom of the glass vial. The larger masses and small crushed particles were weighed so that 5 to 10 mg of the masses and the crushed particles were placed into separate 1.5 ml conical polypropylene tubes, then 1 ml of phosphate buffered saline was added. The masses or particles were disbursed into the liquid. One hour after disbursing the samples, the contents of the tubes were mixed again and then the tubes centrifuged for 5 minutes at 5,000 rpm (Eppendorf Centrifuge, Model No. 5415). A sample of 0.1 ml was removed for assay and replaced with 0.1 ml of PBS. This procedure was repeated to take samples at 65 hours. At 98 hours and each time point thereafter, the full volume of release medium was removed and replaced with a fresh 1 ml of PBS.

Samples were analyzed for protein content with the microassay procedure for microtiter plates (Bio-Rad protein assay, based on the method of Bradford; Coomassie Brilliant Blue Dye, Cat. No. 500-0006) with 96 well microtiter plates. Standards contained 5 to 60 μg/ml of BSA. Standards and samples were added to the wells in a volume of 0.16 ml first, then 40 μl of dye was added to each well with mixing before reading the absorbance at 630 nm. Standard curves were constructed from absorbances corrected for the blank values in the absence of added protein (BSA). Protein concentrations of the samples were calculated from the standard curve that was based on the same lot of BSA and prepared on the basis of weight of BSA to total volume (w/v). The values for the protein released at various times were adjusted by determining differences in the protein concentration of the lyophilized BSA that was weighed and placed in solution from the BSA taken directly from the bottle of the commercial supplier (USB, Amersham Life Sciences, Cat. No. 10868) and placed in solution.

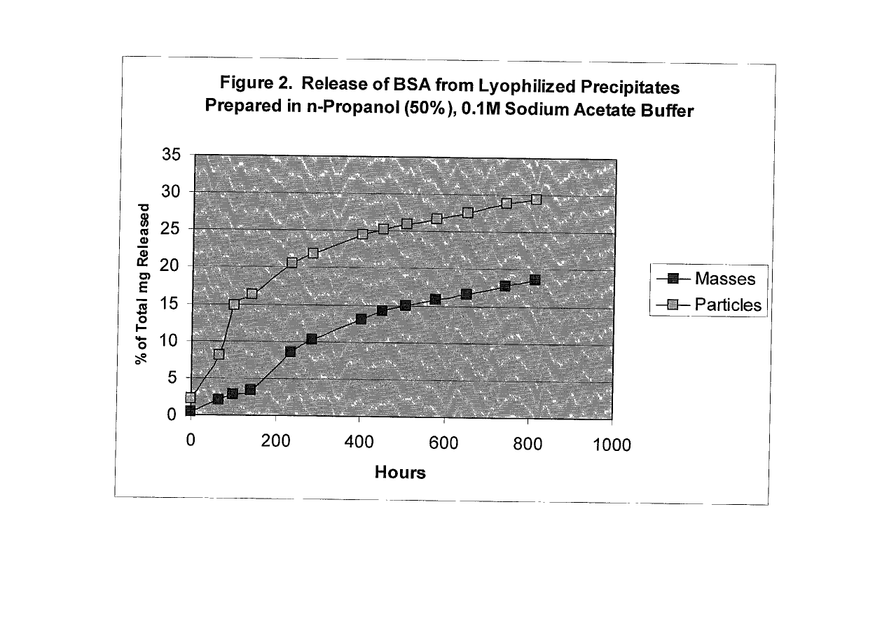

The results of the controlled release are shown in FIG. 2 [nP, represents n-propanol]. As can be seen, there is no burst effect and the release is essentially linear. The smaller particles with a large surface area to mass ratio release at a faster rate. There appears to be a slightly faster rate of release during the first hours of release (FIG. 2). This faster release rate can be eliminated by preincubating the samples in medium prior to use.

### EXAMPLE 3

Release of BSA was measured up to 811 hours from samples of lyophilized protein precipitated from alcohol/aqueous solution. This example briefly describes sample preparation and analytical methodology and presents results showing controlled release of BSA. The release is affected by the specific alcohol used, the buffer used, and the particle size of the precipitated and lyophilized protein.

Solutions of BSA (USB, Amersham Life Sciences, Cat. No. 10868) at 5% (w/w) were prepared in 0.01 M acetate buffer using an equivalent volume of 0.005 M sodium acetate and 0.005 M acetic acid. The pH was approximately 5. The t-butyl alcohol was added to a concentration of 40% (v/v). After overnight equilibration at room temperature, the supernatant was removed and the precipitate frozen at −20 C and brought to −70 C before lyophilization. The surface upon which the vials were placed and the lyophilizer chamber was precooled to maintain the samples frozen during the lyophilization procedure. The sample was lyophilized for 5 hours. The time of lyophilization can be longer or shorter depending on the volume to be lyophilized. The lyophilized sample was divided into several pieces with a spatula. The pieces were divided into small particles by crushing the pieces against the wall and bottom of the glass vial. The larger masses and small crushed particles were weighed so that 5 to 10 mg of the masses and the crushed particles were placed into separate 1.5 ml conical polypropylene tubes, then 1 ml of phosphate buffered saline was added. The masses or particles were disbursed into the liquid. One hour after disbursing the samples, the contents of the tubes were mixed again and then the tubes centrifuged for 5 minutes at 5,000 rpm (Eppendorf Centrifuge, Model No. 5415). A sample of 0.1 ml was removed for assay and replaced with 0.1 ml of PBS. This procedure was repeated to take samples at 65 hours. At 98 hours and each time point thereafter, the full volume of release medium was removed and replaced with a fresh 1 ml of PBS.

Samples were analyzed for protein content with the microassay procedure for microtiter plates (Bio-Rad protein assay, based on the method of Bradford; Coomassie Brilliant Blue Dye, Cat. No. 500-0006) with 96 well microtiter plates. Standards contained 5 to 60 μg/ml of BSA. Standards and samples were added to the wells in a volume of 0.16 ml first, then 40 μl of dye was added to each well with mixing before reading the absorbance at 630 nm. Standard curves were constructed from absorbances corrected for the blank values in the absence of added protein (BSA). Protein concentrations of the samples were calculated from the standard curve that was based on the same lot of BSA and prepared on the basis of weight of BSA to total volume (w/v). The values for the protein released at various times were adjusted by determining differences in the protein concentration of the lyophilized BSA that was weighed and placed in solution from the BSA taken directly from the bottle of the commercial supplier (USB, Amersham Life Sciences, Cat. No. 10868) and placed in solution.

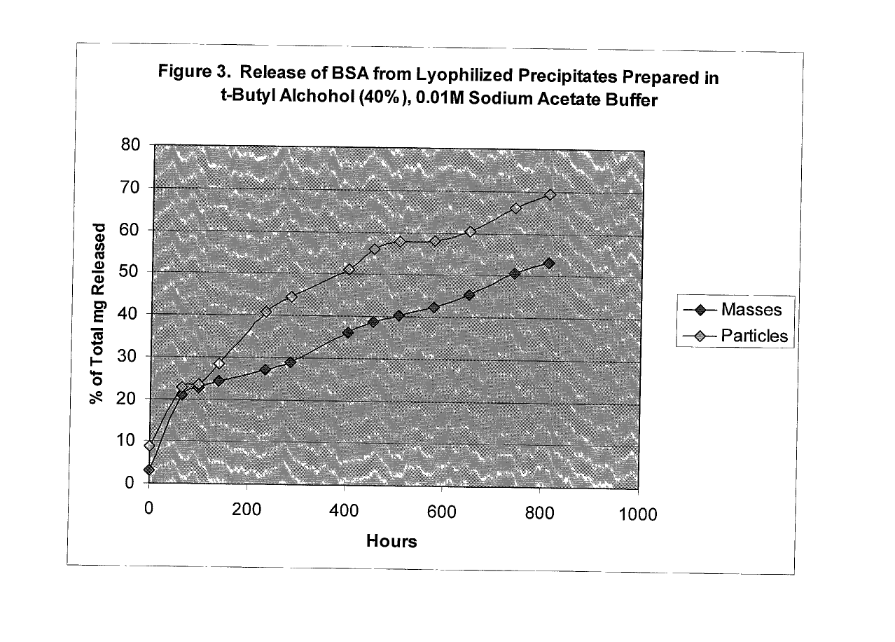

The results of the controlled release are shown in FIG. 3 [tBA, represents t-butyl alcohol]. As can be seen, there is no major burst effect and the release is essentially linear after the first hours. The smaller particles with a large surface area to mass ratio release at a faster rate. There appears to be a slightly faster rate of release during the first hours of release (FIG. 3). This faster release rate can be eliminated by preincubating the samples in medium prior to use.

### EXAMPLE 4

Release of BSA was measured up to 811 hours from samples of lyophilized protein precipitated from alcohol/aqueous solution. This example briefly describes sample preparation and analytical methodology and presents results showing controlled release of BSA. The release is affected by the specific alcohol used, the buffer used, and the particle size of the precipitated and lyophilized protein.

Solutions of BSA (USB, Amersham Life Sciences, Cat. No. 10868) at 5% (w/w) were prepared in 0.1 M acetate buffer using an equivalent volume of 0.05 M sodium acetate and 0.05 M acetic acid. The pH was approximately 5. The alcohol t-butyl alcohol was added to a concentration of 40% (v/v). After overnight equilibration at room temperature, the supernatant was removed and the precipitate frozen at −20 C and brought to −70 C before lyophilization. The surface upon which the vials were placed and the lyophilizer chamber was precooled to maintain the samples frozen during the lyophilization procedure. The sample was lyophilized for 5 hours. The time of lyophilization can be longer or shorter depending on the volume to be lyophilized. The lyophilized sample was divided into several pieces with a spatula. The pieces were divided into small particles by crushing the pieces against the wall and bottom of the glass vial. The larger masses and small crushed particles were weighed so that 5 to 10 mg of the masses and the crushed particles were placed into separate 1.5 ml conical polypropylene tubes, then 1 ml of phosphate buffered saline was added. The masses or particles were disbursed into the liquid. One hour after disbursing the samples, the contents of the tubes were mixed again and then the tubes centrifuged for 5 minutes at 5,000 rpm (Eppendorf Centrifuge, Model No. 5415). A sample of 0.1 ml was removed for assay and replaced with 0.1 ml of PBS. This procedure was repeated to take samples at 65 hours. At 98 hours and each time point thereafter, the full volume of release medium was removed and replaced with a fresh 1 ml of PBS.

Samples were analyzed for protein content with the microassay procedure for microtiter plates (Bio-Rad protein assay, based on the method of Bradford; Coomassie Brilliant Blue Dye,Cat. No. 500-0006) with 96 well microtiter plates. Standards contained 5 to 60 μg/ml of BSA. Standards and samples were added to the wells in a volume of 0.16 ml first, then 40 μg of dye was added to each well with mixing before reading the absorbance at 630 nm. Standard curves were constructed from absorbances corrected for the blank values in the absence of added protein (BSA). Protein concentrations of the samples were calculated from the standard curve that was based on the same lot of BSA and prepared on the basis of weight of BSA to total volume (w/v). The values for the protein released at various times were adjusted by determining differences in the protein concentration of the lyophilized BSA that was weighed and placed in solution from the BSA taken directly from the bottle of the commercial supplier (USB, Amersham Life Sciences, Cat. No. 10868) and placed in solution.

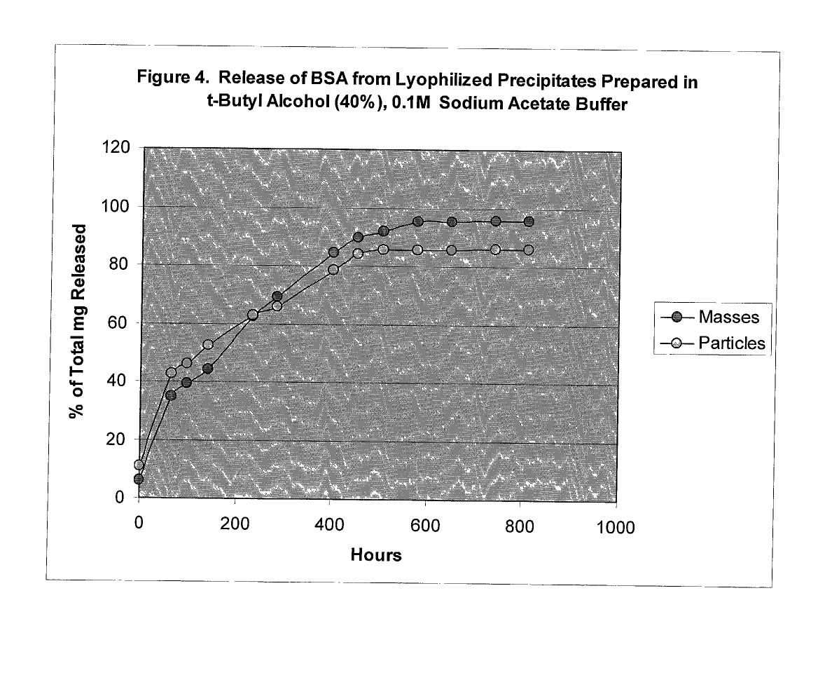

The results of the controlled release are shown in FIG. 4 [tBA, represents t-butyl alcohol]. As can be seen, there is no major burst effect and the release is essentially linear after the first hours. The smaller particles with a large surface area to mass ratio release at a faster rate. There appears to be a slightly faster rate of release during the first hours of release (FIG. 4). This faster release rate can be eliminated by preincubating the samples in medium prior to use.

Comparison of the Release Data.

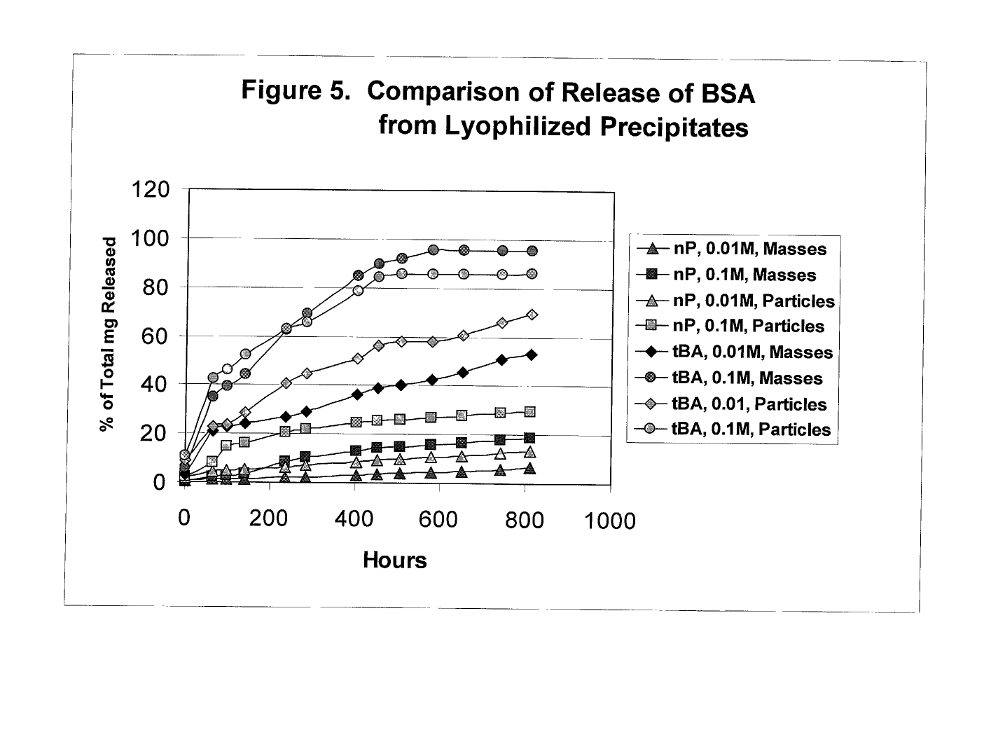

A comparison of the release kinetics for all the samples are shown together on a single chart (FIG. 5). It can be seen that the various samples have release kinetics that will last for a wide variety of periods: from 500 hrs (21 days) to about 10,000 hrs (over 1 year). Combinations of the samples can produce release kinetics with a variety of release rates at different times. The small particles exhibited faster release rates except for the most rapidly releasing preparation (FIG. 5; FIG. 4; 0.1 M acetate; t-butyl alcohol, 40%). The results demonstrate that salt concentrations and the type of alcohol can modify the release rates extensively.

General Materials and Methods for Examples 5-13

(i) Materials

Bovine Serum Albumin (Cat. #10868, lot #107331, USB)

Human Serum Albumin (Cat. #10878, lot #103077, USB)

Albumin (Human) 25% Solution: Immuno-U.S., Inc. (NDC 64193-228-05, lot #628808)

Albumin (Human) 25% Solution: Alpha Therapeutic (Cat #521302, lot #NG9856A)

Interferon-α001 (PBL) 0.94 mg/ml in Tris Buffer [see also U.S. Pat. Nos. 5,789,551, 5,869,293, 6,001,589, 6,299,870, 6,300,474]

Interferon-α012 (PBL) 1.38 mg/ml in Tris Buffer

Tris Buffer (20 mm Tris, 200 mm NaCl, 6% glycerol, pH 7-8)

Interferon ELISA (PBL product #41110)

PBS (Dulbecco's Phosphate Buffered Saline, Cat. #8537, Sigma Chemical Co., or Cat. #14198-144, Gibco-BRL)

(ii) Methods

Protein Precipitation.

Proteins were precipitated at ambient temperature (about 24° C.) by one of two basic procedures: the organic addition method or the acid addition method. With the organic addition method, the protein solution was prepared in aqueous solution and an organic component added to precipitate the protein. (Alternatively, an aqueous solution containing protein can be added to the organic solution.) For the acid addition method, a portion of the organic component was added to the protein solution under conditions that do not precipitate the protein. Precipitation was initiated by adding an acidified solution concurrent with or after addition of organic components to the protein solution. Unless otherwise stated in the legends, deionized water was used to dilute formulation reagents. HSA stock solutions were made by diluting 25% source material to 1% final concentration, and data presented were obtained using Immuno-U.S. Human Serum Albumin.

Adjustment of pH.

Because organic solvent hinders the ability to accurately measure pH, the pH specified for any formulation refers to the pH of the (aqueous) solution prior to addition of the organic component. In the case of the organic addition method, the pH of an aqueous protein solution was adjusted to the desired pH just prior to adding the organic component. To make the same formulation by the acid addition method, an equivalent amount of acid was added in the final step rather than prior to addition of the organic solvent.

Maturation Procedures.

The maturation period began after addition of the final formulation component to initiate precipitation and ended when centrifugation was initiated to separate precipitate from supernatant. The release properties of the precipitate depend on the maturation time as well as the conditions of the formulation during this period. Temperature was ambient, about 24° C. unless otherwise noted. Formulations were mixed by vessel rotation, stirred in tubes or in vials containing a magnetic stir-bar, or mixed initially and left undisturbed. In addition, during the maturation period some formulations were drawn through a syringe needle one to three times toward the end of the maturation period.

Wash Procedures.

The first steps in washing precipitates were to 1) separate the precipitate from supernatant by centrifugation, 2) remove as much supernatant as possible without disturbing the precipitate, and 3) re-suspend the precipitate in PBS/0.01% thimerosal. Precipitates were harvested and washed (PBS/0.01% thimerosal) once or twice by centrifugation for 2-5 min at 3,000 to 15,000 rpm in a Beckman or Eppendorf microcentrifuge. A sample of the harvested supernatant was diluted 10-fold in PBS/0.01% thimerosal to prevent (through dilution of organic and acid) further precipitation of protein in the diluted supernatant. If the release experiment was to begin immediately, the last harvested wash sample was labeled as the zero time sample and the resuspended preparations placed in an incubator at 37° C. at which temperature release was measured for all samples. Alternatively, the sample could be lyophilized without resuspension after initial harvest or after wash cycles.

Lyophilization.

Precipitates to be lyophilized without washing were cooled to 0-4° C., then sequentially at −20° C., -70° C., and -135° C., at least 15 min at each temperature. Precipitates to be lyophilized after washing with PBS/0.01% thimerosal were frozen only at −20° C. Formulations were lyophilized in a Virtis Freezemobile 6 equipped with a Unitop 100 SM Bulk/Stoppering Chamber. The lyophilizer shelf was pre-cooled with dry ice before transferring vials from the freezer to the shelf. Vials were lyophilized for 2-5 hr at <400 mTorr.

Release Measurements.

Sufficient PBS to make a total volume of 1 ml of release medium (PBS/0.01% thimerosal) was added to the washed and/or lyophilized precipitates. Each precipitate was suspended in release medium (PBS/0.01% thimerosal) before placing the release sample in a 37° C. incubator to begin measuring release of the proteins. At selected time intervals, tubes containing the samples with the release medium were removed from the incubator and centrifuged for 2-5 min at 3,000 to 15,000 rpm. The majority of the medium containing the released protein in the supernatant, usually about 0.9 ml, was removed and replaced with an equal volume of fresh PBS/0.01% thimerosal.

Sample Analysis.

Albumin samples were assayed as is or diluted with PBS/0.01% thimerosal to the range of the Bio-Rad Protein Assay (Bio-Rad Labs). Stock solutions diluted from the source albumin raw material in the formulations were used as assay standards. Interferon samples, as is or diluted with PBS/0.01% thimerosal, were assayed by ELISA (PBL Biomedical Laboratories, product # 41110).

Calculations.

The cumulative quantity of analyte released at each sample time was calculated by adding the amount released in the n^{th} sample to the sum of the quantities released in the previous samples. The quantity released in the nth sample was corrected for the residual quantity left in the tube from the previous sample since typically 0.9 ml of the total volume of 1.0 ml was collected at each sample interval. Cumulative quantities released were plotted as the mass released or as a percentage of the calculated total analyte present in the precipitate at the start of incubation at 37° C. (start of the release). The total analyte present in the precipitates at the start of the release was calculated by subtracting the quantity of analyte recovered in the supernatant and wash samples from the original amount of analyte added to the formulation.

### EXAMPLE 5

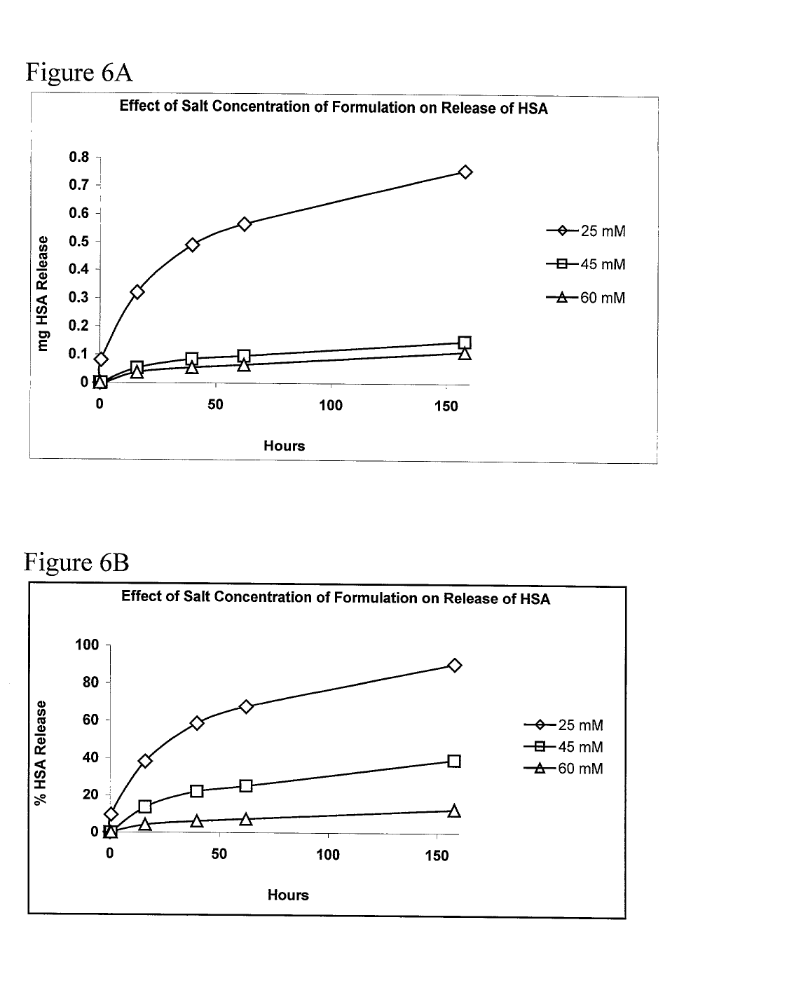

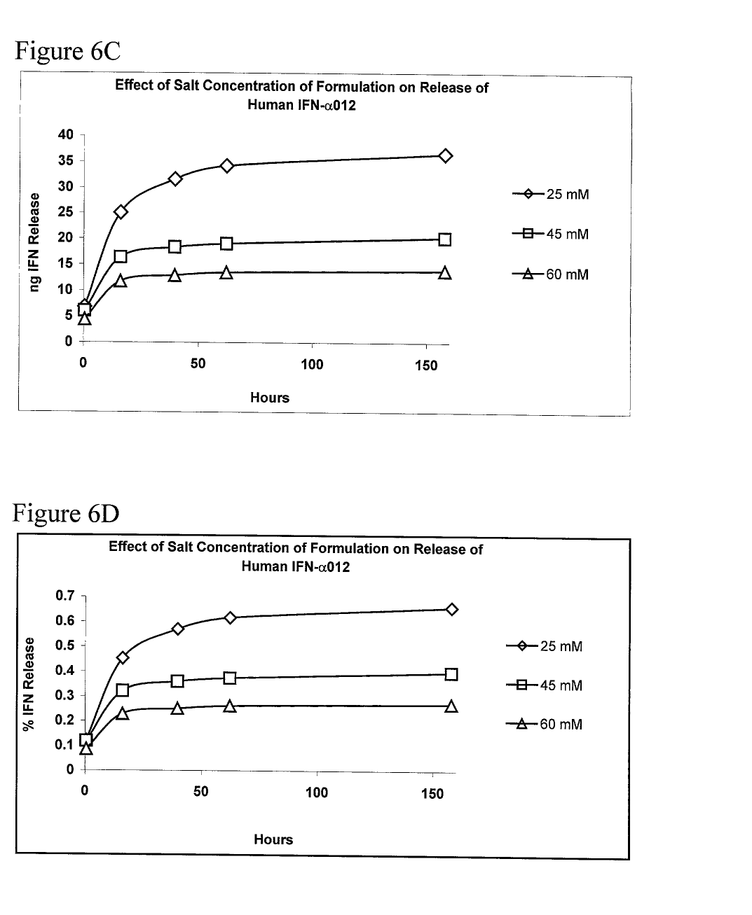

As an embodiment of the sustained release, the release of HSA and human IFN-α012 as a function of sodium acetate concentration was evaluated as shown in FIG. 6. Solution I consisted of 9.0 mg of HSA (Immuno-U.S.) and 10 μg of IFN-α012 in 40% (w/w) n-propanol (0.364 g n-propanol) in H₂O for a total weight of 0.91 g. The various Solution II compositions consisted of various quantities of sodium acetate (1 M, pH 6.3) and deionized water and 0.040 g n-propanol to make solutions of 40% n-propanol and 250, 450, and 600 mM final sodium acetate concentrations with a total volume of 0.10 g. Solution II (0.10 g) was added to Solution I (0.91 g) with stirring to yield a final 1.01 g of each formulation. The final 1.01 g formulations containing 40% n-propanol and 25, 45, and 60 mM concentrations of sodium acetate were stirred in 2 ml glass vials for 6 hr at 24° C. and passed through 25G syringe needles just prior to separating supernatants from precipitates. The quantity of HSA and IFN-α012 in washed precipitates was determined as described in Materials and Methods. Release was performed in PBS/0.01% thimerosal. As can be seen the early burst phase of the sustained release and the rate of release of HSA and human IFN-α012 can be altered by the sodium acetate concentration. Higher sodium acetate concentration decreased the burst rate (0-24 hour period) extensively and decrease the rate of release of the HSA and human IFN-α012 (FIGS. 6A-D). Release continued after analysis period of about 7 days. The burst phase for release of human IFN-α012 was especially sensitive to the sodium acetate concentration. The release was monitored for about 160 hrs (over six days).

### EXAMPLE 6

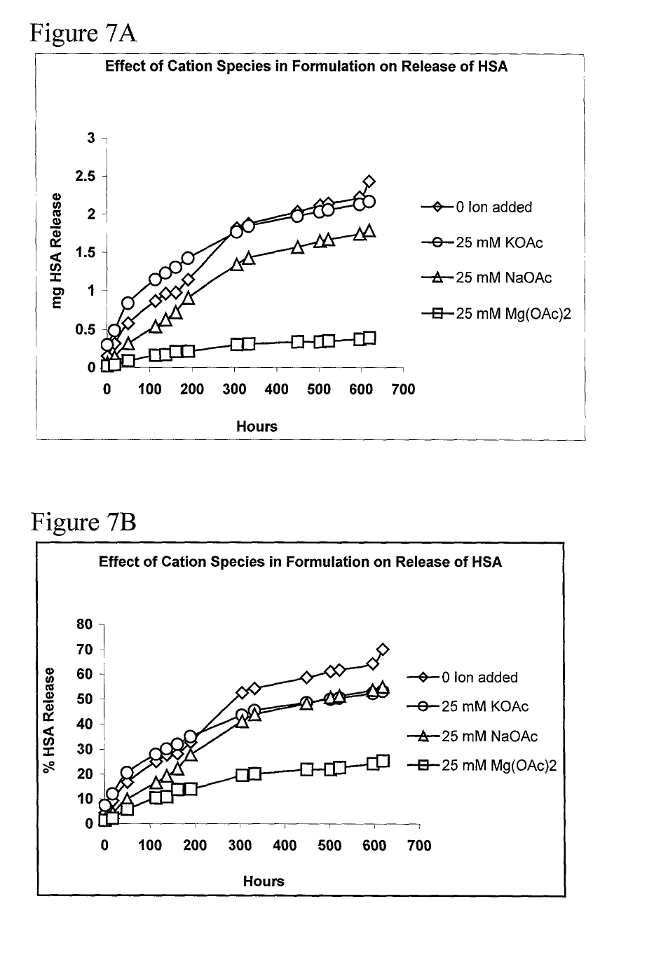

Effect of cation species in formulation on release of HSA is shown in FIGS. 7A,B. Solution I consisted of 8.1 mg of HSA (Immuno-U.S.) in 40% (w/w) n-propanol in deionized water in a total volume of 0.91 ml. The various Solution II compositions consisted of adding none or 0.025 ml of various salt stocks (each at 1 M cation concentration, pH 6.3) to deionized water followed by n-propanol to make solutions 40% (w/w) n-propanol and 250 mM final cation concentration in a total volume of 0.10 ml. Solution 11 (0.10 ml) was added to 0.91 ml of Solution I with stirring to give a final 1.01 ml formulation having 40% (w/w) n-propanol. The final 1.01 ml formulations containing 40% n-propanol and no or 25 mM concentrations of potassium, sodium or magnesium acetate were stirred in 2 ml glass vials for 6 hr at 24° C. prior to separating supernatants from precipitates. The quantity of HSA in washed precipitates was determined as described in Materials and Methods. Release was performed in PBS/0.01% thimerosal. The burst rate in the first 24 hours was reduced substantially by sodium and even further by magnesium in the formulation. Furthermore, the release rate can be increased or reduced by use of the various acetates. Extended release rates of over 25 days (over 600 hrs) were achieved with all these formulations. Release was projected to continue beyond the time measured by the graphs (FIGS. 7A,B). The release was monitored for over 600 hrs or 25 days.

### EXAMPLE 7

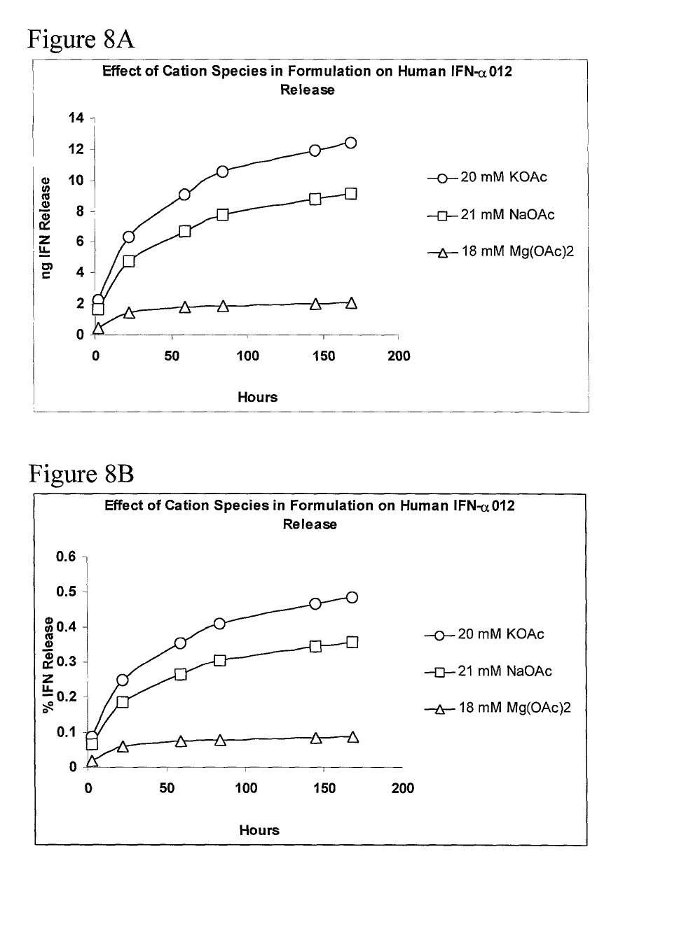

Effect of cation species in formulation on release of human IFN-α012 is shown in FIGS. 8A,B. Solution I consisted of 45 mg of HSA (Immuno-U.S.) and 5.44 μg IFN-α012 in 40% (w/w) n-propanol in deionized water in a total volume of 4.55 ml. The various Solution II compositions consisted of adding 36 μl of 0.1 M acetic acid (to compensate for the buffer capacity of the HSA solution) and 0.250 g of potassium, sodium or magnesium acetate solution (each at pH 6.3) to 0.314 g of deionized water and 0.400 g of n-propanol to make solutions of 40% (w/w) n-propanol and 250 mM final acetate concentration in a total weight of 1 g. The potassium acetate solution was made with 0.980 g potassium acetate, 10.061g water and 0.274 ml 1 M acetic acid. The sodium acetate solution was made with 0.823 g sodium acetate, 10.056 g water and 0.245 ml 1 M acetic acid. The magnesium acetate solution was made with 2.144 g magnesium acetate, 10 g water and 0.200 ml 1 M acetic acid. Solution 11 (0.50 ml) was added to 4.55 ml of Solution I with stirring to give a final 5.05 ml formulation having 40% (w/w) n-propanol. The final formulations were stirred in 50 ml conical tubes for 6 hr at 24° C., the precipitates washed with 5 ml of PBS/0.01% thimerosal, then suspended in 5 ml PBS/0.01% thimerosal, then split into two individual 2.5 ml samples prior to separating supernatants from precipitates. Release data are from the precipitates from one 2.5 ml portion of the formulation. The salt concentrations in the formulations were 21 mM NaOAc, 20 mM KOAc and 18 mM Mg(OAc)₂ in the respective solutions. The quantity of IFN-α012 in washed precipitates was determined as described in Materials and Methods. Release was performed in PBS/0.01% thimerosal. The burst rate can be reduced extensively from potassium to sodium and to magnesium acetate in that order (FIG. 8). In addition, the overall rate of release can be modulated by these salts: the rate of release of IFN-α012 is fastest with potassium acetate, less with sodium acetate and slowest with magnesium acetate (FIG. 8). The release was monitored for about 170 hrs or seven days.

### EXAMPLE 8

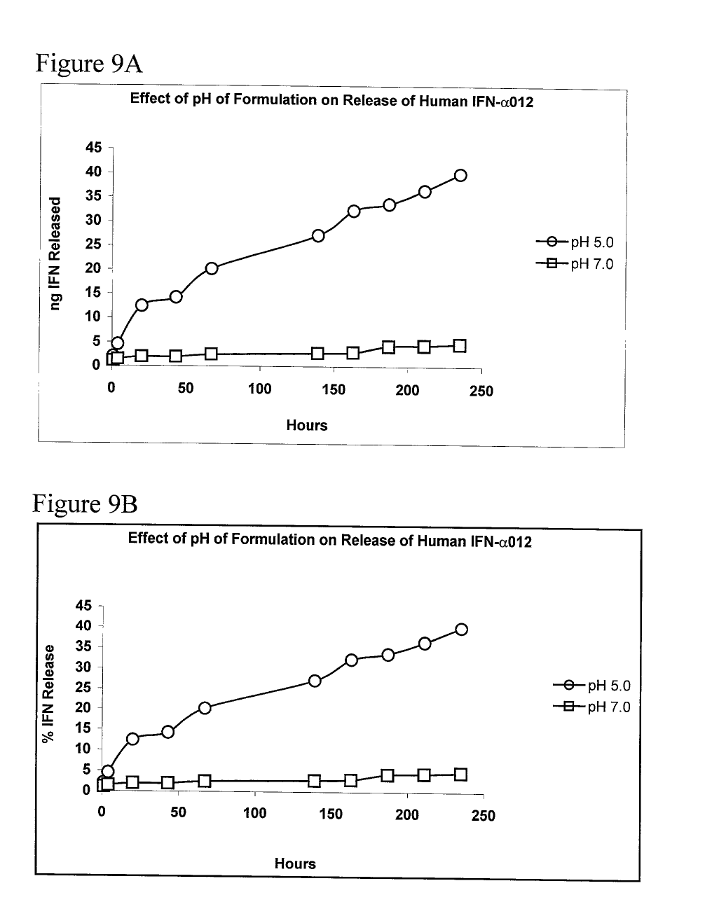

Effect of pH of formulation on release of human IFN-α012 is shown in FIG. 9. Acetic acid (0.1 M) was used to adjust 5% HSA stock solutions to pH 5.0 or pH 7.0. Solution I consisted of 10 mg of HSA (Alpha Therapeutic) from either pH 5.0 or pH 7.0 HSA stock solutions, 6.83 μg IFN-α012 and additional water to a total weight of 0.6 g. The final formulations were prepared by adding 0.4 g of n-propanol to Solution I with stirring to yield a concentration of 40% (w/w) n-propanol. Final 1 g formulations were stirred in 2 ml glass vials for 24 hr at 24° C. prior to separating supernatants from precipitates. The quantity of IFN-α012 in washed precipitates was determined as described in Materials and Methods. Release was performed in PBS/0.01% thimerosal. The burst was modest at both pH 5.0 and pH 7.0 and was remarkably approaching linearity at both pH values (FIG. 9). The lower pH increased the rate of release extensively. Relatively little or no overall burst effect was evident. The release was monitored for about 240 hrs or ten days.

### EXAMPLE 9

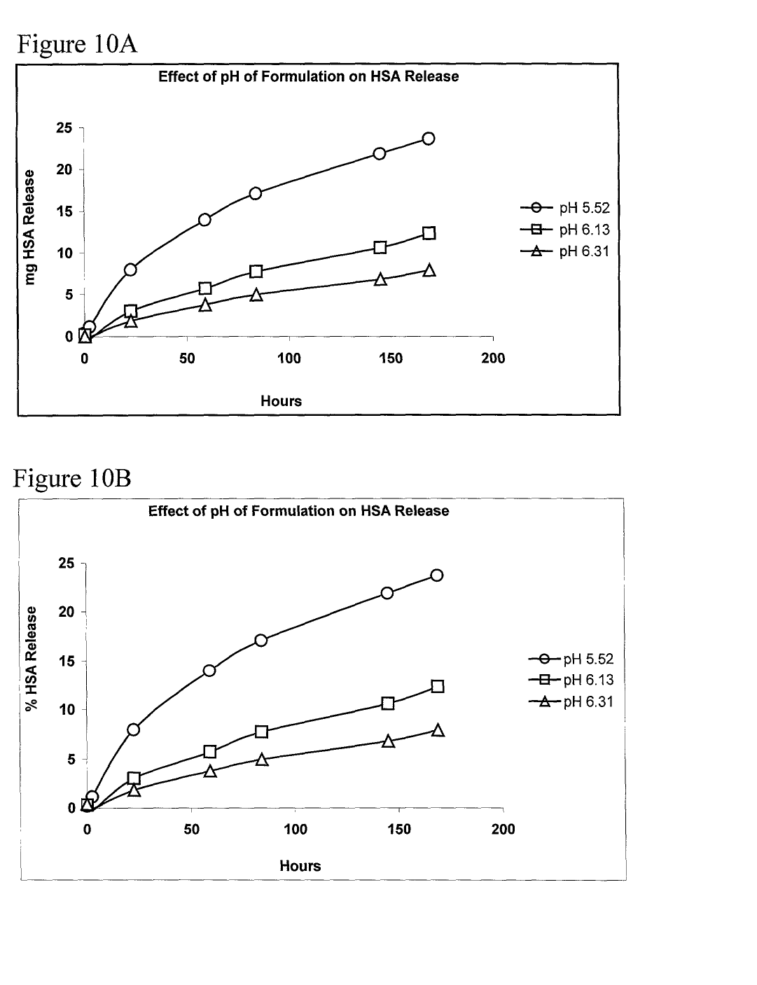

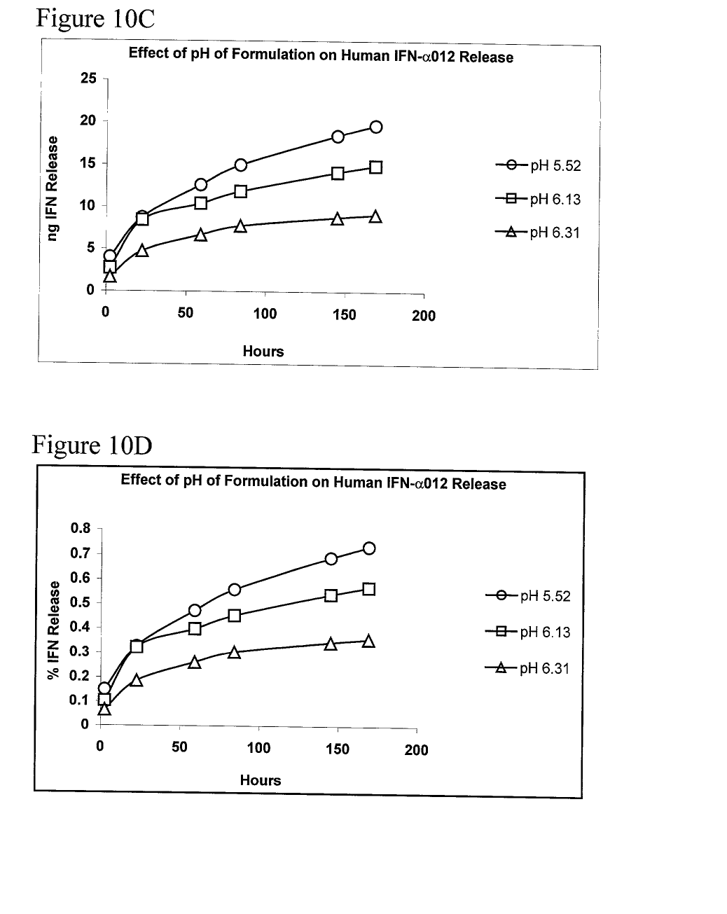

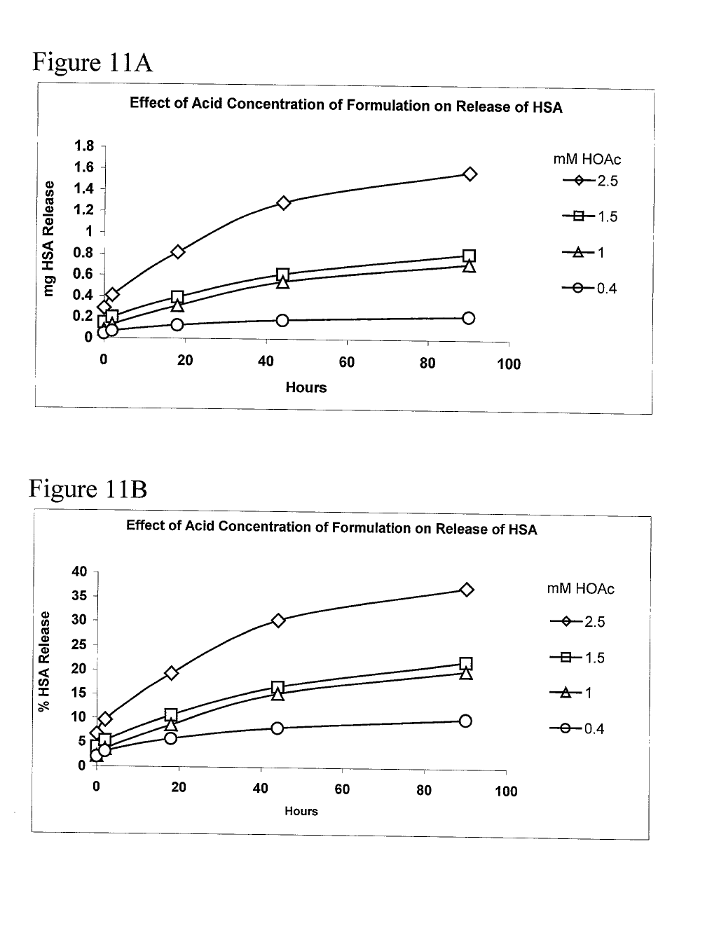

Effect of pH of formulation on release of HSA and human IFN-α012 is shown (FIGS. 10A-D). Solution I consisted of 45 mg of HSA (Immuno-U.S.) and 5.44 μg IFN-α012 in 40% (w/w) n-propanol in deionized water in a total volume of 4.55 ml. Solution II compositions were prepared as follows. Solution IIa: 1.55 ml of 1 M acetic acid was added to 0.82 g anhydrous sodium acetate and 10 g deionized water to adjust pH of this Solution A to 5.52; then 0.036 ml of 0.1 M acetic acid was added to 0.250 g of Solution A to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. Solution IIb: 0.40 ml of 1 M acetic acid was added to 0.82 g anhydrous sodium acetate and 10 g of deionized water to adjust pH of this Solution B to 6.13; then 0.036 ml of 0.1 M acetic acid was added to 0.250 g of Solution B to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. Solution IIc: 0.245 ml of 1 M acetic acid was added to 0.823 g anhydrous sodium acetate and 10.056 g deionized water to adjust pH of this Solution C to 6.31; then 0.036 ml of 0.1 M acetic acid was added to 0.250 g of Solution C to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. To prepare the final formulations, 0.50 ml from Solutions Ia, IIb, or IIc was added to 4.55 ml of Solution I with stirring to yield three 5.05 ml formulations having 40% (w/w) n-propanol and pH 5.52, pH 6.13 or pH 6.31, respectively. Final formulations were stirred in 50 ml conical tubes for 6 hr at 24° C., then split into two individual 2.52 ml samples prior to separating supernatants from precipitates. Release data is from one 2.52 ml portion of the formulation. The amount of IFN-α012 in washed precipitates was determined as described in Materials and Methods. Release was performed in PBS/0.01% thimerosal. The overall burst was minimal at all pH values (pH 5.52, pH 6.13 and pH 6.31 (FIGS. 10A,B) for HSA, but slightly greater for human IFN-α012 (FIGS. 10C,D). The rate of release of both HSA and human IFN-α012 was increased by lowering the pH in all cases (FIGS. 10A-D) as also shown in FIG. 9. Of note is that small changes in the pH can modulate the rate of release and that overall changes in release are the same for HSA and IFN-α012.

### EXAMPLE 10

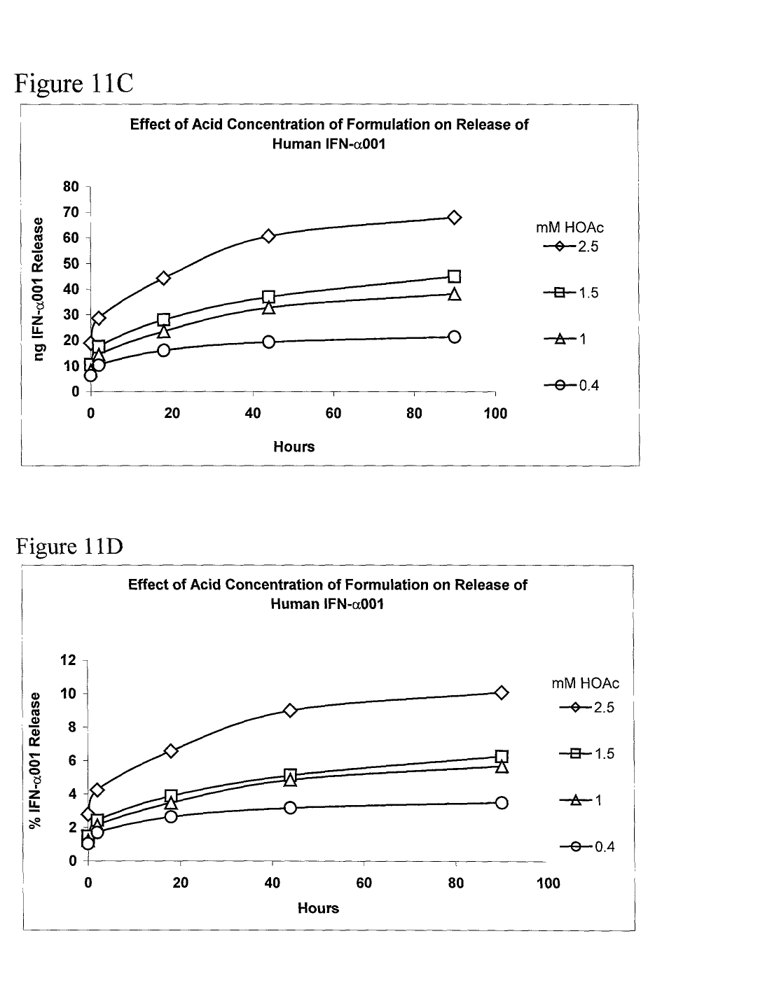

Effect of acid concentration of formulation on release of HSA and human IFN-α001 from precipitates formed in the presence of 25 mM sodium acetate is shown in FIG. 11. Solution I consisted of 8.1 mg of HSA (Immuno-U.S.) and 0.92 μg IFN-α001 in 40% (w/w) n-propanol in deionized water in a total volume of 0.9 ml. Several Solution II formulations, Ia, IIb, IIc and IId, were prepared consisting of 0.004, 0.010, 0.015 and 0.025 ml of 0.1 M acetic acid, respectively, in 40% (w/w) n-propanol in deionized water. Solution III consisted of 1 M sodium acetate and 40% (w/w) n-propanol in deionized water in a total volume of 0.025 ml. Several Solution IV formulations, IVa, IVb, IVc and IVd, were prepared consisting of 0.071, 0.065, 0.060 and 0.050 ml of 40% (w/w) n-propanol, respectively, in deionized water. In preparing the final formulations, Solutions Ia, IIb, IIc and IId were matched with Solutions IVa, IVb, IVc and IVd, respectively. Solutions II, III and IV were mixed together then Solution I added rapidly to the mixture to give a final 1 ml formulation. This yielded a formulation having a final concentration of 25 mM sodium acetate, 40% (w/w) n-propanol and the final acetic acid concentrations indicated on the Figure. Formulations were stirred in 2 ml glass vials for 6 hr at 24° C. prior to separating supernatants from precipitates. After washing, precipitates were lyophilized 4 hr at <400 mTorr. The amount of HSA in washed precipitates was determined as described in Materials and Methods. Release was performed in PBS/0.01% thimerosal. The burst is increased by increased quantity of acetic acid comparable to the increase in burst on decrease of pH as seen in FIGS. 9 and 10. Furthermore, the rate of release increases with the quantity of acid also comparable to the increase in rate of release with decrease in pH as seen in FIGS. 9 and 10. The release of HSA and human IFN-α001 was monitored for about 90 hrs (FIGS. 11A-D).

### EXAMPLE 11

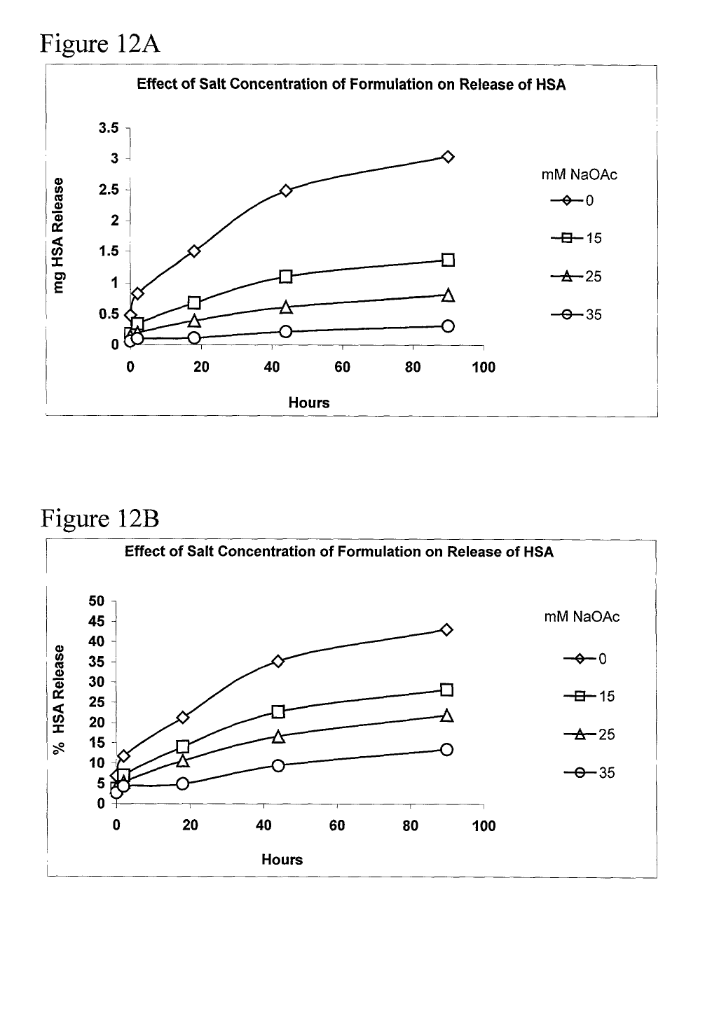

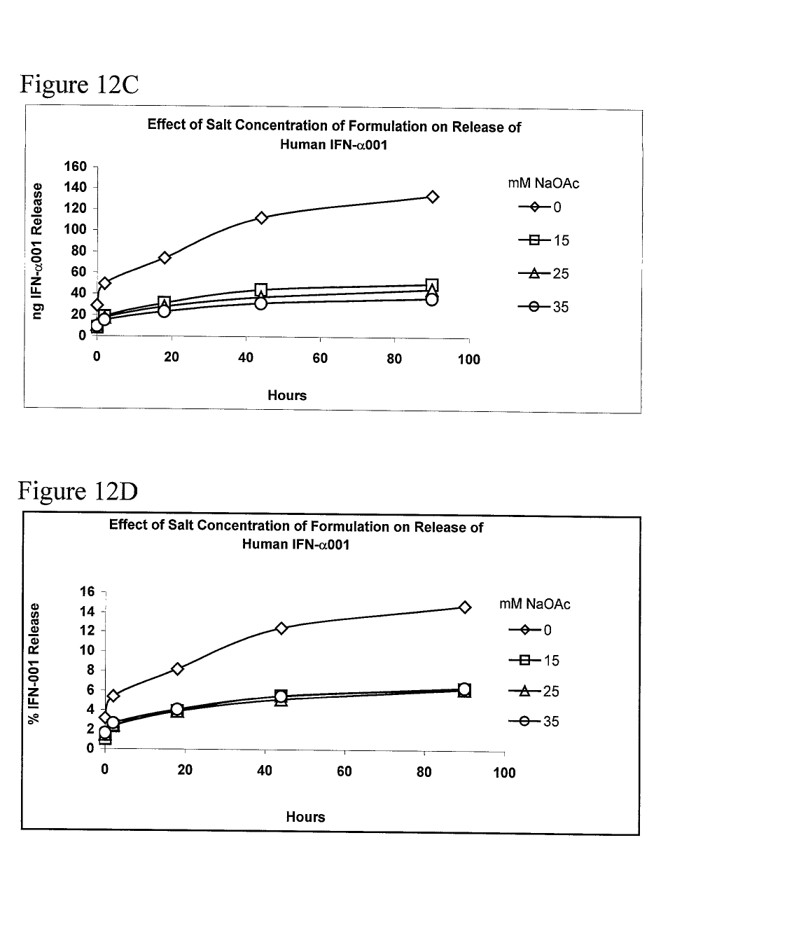

Effect of salt concentration of formulation on release of HSA and human IFN-α001 from precipitates formed in the presence of 1.5 mM acetic acid is shown in FIG. 12. Solution I consisted of 8.1 mg of HSA (Immuno-U.S.) and 0.92 μg IFN-α001 in 40% (w/w) n-propanol in deionized water in a total volume of 0.9 ml. Solution II consisted of 0.1 M acetic acid and 40% (w/w) n-propanol in deionized water in a total volume of 0.015 ml. Several Solution III formulations, IIIa, IIIb, IIIc and IIId, were prepared consisting of 0, 0.015, 0.025 and 0.035 ml of 1 M sodium acetate, respectively, in 40% (w/w) n-propanol in deionized water. Several Solution IV formulations, IVa, IVb, IVc and IVd, were prepared consisting of 0.085, 0.070, 0.060 and 0.050 ml of 40% (w/w) n-propanol, respectively, in deionized water. In preparing the final formulations, Solutions IIa, IIIb, IIIc and IIId were matched with Solutions IVa, IVb, IVc and IVd, respectively. Solutions II, III and IV were mixed together then Solution I added rapidly to the mixture to give a final 1 ml formulation. This yielded a final concentration of 1.5 mM acetic acid, 40% (w/w) n-propanol (w/w) and the final sodium concentrations indicated on the Figure. Formulations were stirred in 2 ml glass vials for 6 hr at 24° C. prior to separating supernatants from precipitates. After washing, precipitates were lyophilized 4 hr at <400 mTorr. The amounts of HSA and IFN-α001 in washed precipitates were determined as described in Materials and Methods. Release was performed in PBS/0.01% thimerosal. Increased salt concentration minimizes the burst and reduces the rate of release of HSA (FIGS. 12A, B) and IFN-α001 (FIGS. 12C, D). Much of the burst can be eliminated by sodium acetate concentrations above 15 mM. The release was monitored for about 90 hrs.

### EXAMPLE 12

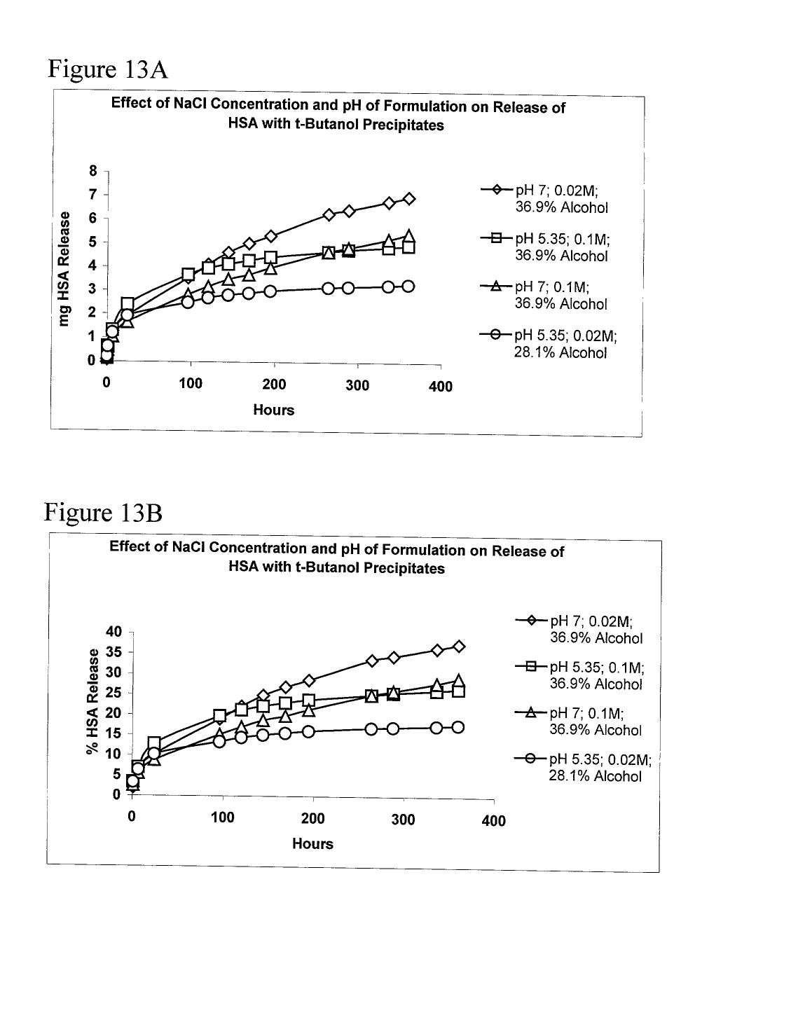

Effect of salt concentration and pH of formulation on release of HSA with tertiary butanol precipitates is shown in FIG. 13. Acetic acid (0.1 M) was used to adjust 5% HSA stock solutions to pH 5.35 or 7.0. Solution I consisted of 18.0 mg of HSA (Alpha Therapeutic) from the pH 5.35 or pH 7.0 5% stock solution, 1.0 μg IFN-AO12 and deionized water bringing the total solution weight to 0.375 g. To prepare Solutions IIa and IIb with NaCl concentrations of 0.02 M and 0.1 M, respectively, sufficient deionized water was added to 0.021 and 0.0043 ml of a 3.75 M NaCl solution to bring the total weight of each solution to 0.425 g. Both pH 5.35 and pH 7.0 variants of Solution 1 (0.375 g) were added to Solutions Ia and IIb to yield 0.80 g of the various combinations of pH and NaCl concentration as shown in the Figure prior to the addition of 0.31 or 0.47 g of tert-butyl alcohol to yield 28.1% and 36.9% (w/w) tert-butyl alcohol (see summary of the chart legends). Final 1.11-1.27 g formulations were stirred in 2 ml glass vials for 24 hr at 24° C. prior to separating supernatants from precipitates. The amount of HSA in washed precipitates was determined as described in Materials and Methods. Release was performed in PBS/0.01% thimerosal. The pH had very little effect on the burst in the formulations with tertiary butyl alcohol (FIGS. 13A and B). Furthermore, the rate of release of HSA was decreased by decrease in pH in contrast to the formulations with n-propanol (FIGS. 9 and 10). Nevertheless, the overall rate of release of HSA over the 350 hrs of monitoring (FIG. 13). The release rates were more near linearity at pH 7.0 than at pH 5.35.

### EXAMPLE 13

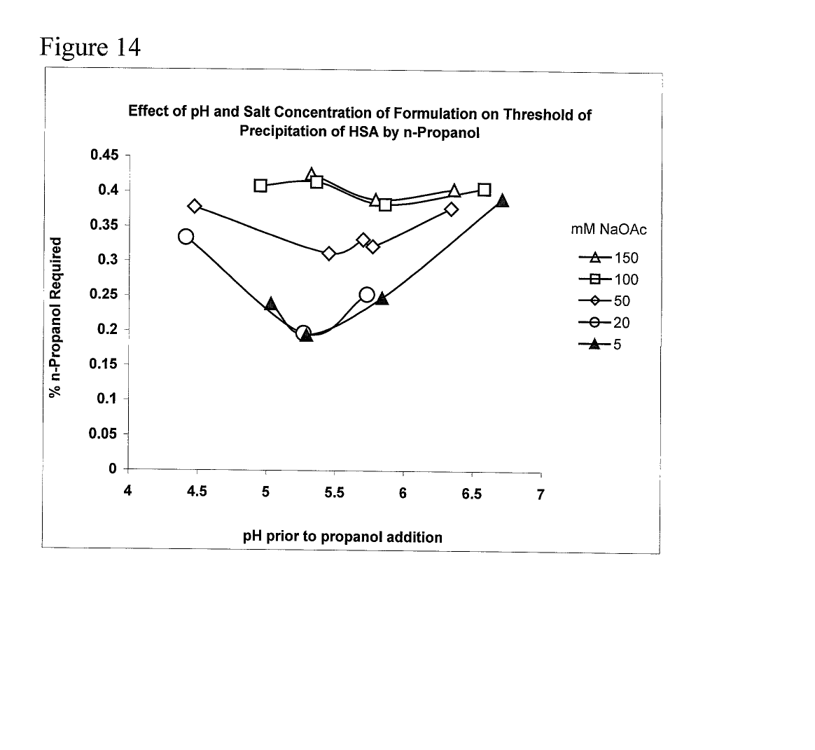

Effect of pH and salt concentration of formulation on threshold of precipitation of HSA by n-propanol is shown in FIG. 14. An 11% (w/w) HSA (USB) solution was dialyzed 3 times for 6 hr each time against 2 L deionized H₂0 in a Pierce Slide alyzer (15 ml capacity, No. 66410, lot #BJ44820B). The final concentration was analyzed by spectrophotometry at 280 nm to be 8.28% (w/w). This solution was diluted to 4% (w/w) with deionized water. Amounts (0.9 g) of 4% HSA were weighed into 2 ml glass vials. Sodium acetate (1 M), acetic acid (1 M), sodium hydroxide (1 M), and water were added in various combinations in a total weight of 0.1 g to yield the final sodium concentrations and pH values measured in 1 g formulations as shown in the Figure. Subsequently, n-propanol was added in about 50 μl increments with stirring, and the point at which initial precipitates were stable (did not re-dissolve with stirring within 5 minutes) was recorded. Connected data points indicate equivalent sodium concentrations at various pH and n-propanol (w/w) concentrations. The threshold of precipitation of HSA can be modified greatly by the sodium acetate concentration. At low sodium acetate concentrations, the least level of n-propanol is required to initiate precipitation of the HSA. These data (FIG. 14) provide general approaches to modulate the formulations.

1. A slow release formulation comprising one or more biologically active molecules from a solid composition prepared by exposure of the biologically active molecules to an organic solvent under conditions wherein a precipitate, lyophilate or crystal is formed.

2. A slow release formulation comprising precipitate, lyophilate or crystals of a polypeptide prepared by exposure of the polypeptide to an organic solvent, which polypeptide is released from the formulation in aqueous solution for a period of at least 7 days.

3. A formulation comprising precipitate, lyophilate or crystals of a biologically active polypeptide prepared by exposure of the polypeptide to a polar protic organic solvent, which formulation, when administered to a patient, releases said polypeptide at a rate providing an average steady state dosage of at least the ED₅₀ for the polypeptide for a period of at least 7 days.

4. The formulation of any of claims 1-3, wherein the organic solvent is an alcohol, an aldehyde, a ketone, a hydrocarbon, an aromatic hydrocarbon, or a mixture thereof.

5. The formulation of any of claims 1-3, wherein the organic solvent is an alcohol or mix of alcohols.

6. The formulation of claim 5, wherein the alcohol is a lower alcohol, or mixture thereof.

7. The formulation of claim 5, wherein the alcohol is selected from the group consisting of methanol, ethanol, isopropanol, n-propanol, n-butanol, isobutanol, and t-butanol, or a mixture thereof.

8. The formulation of any of claims 1-3, wherein the organic solvent is a polar protic solvent.

9. The formulation of any of claims 1-3, wherein the organic solvent is a water-miscible polar protic solvent.

10. The formulation of any of claims 1-3, wherein the biologically active molecules or polypeptides are released from the formulation in aqueous solution at a rate which provides an average steady state dosage of at least the ED₅₀ for the biologically active molecules or polypeptides for a period of at least 50 days.

11. The formulation of any of claims 1-3, wherein the organic solvent(s) are chosen such that, when administered to a patient, the solvent released from the formulation at a rate which remains at least one order of magnitude below the IC₅₀ for deleterious side effects, if any, of the solvent.

12. The formulation of claim 1, wherein biologically active molecule is a polymer selected from the group consisting of a protein, a peptide, a nucleic acid, an oligonucelotide, a carbohydrate, a ganglioside, or a glycan.

13. The formulation of any of claims 2-3, wherein the polypeptide is selected from the group consisting of cytokines, growth factors, somatotropin, growth hormones, colony stimulating factors, , erythropoietin, plasminogen activators, enzymes, T-cell receptors, surface membrane proteins, lipoproteins, clotting factors, anticlotting factors, tumor necrosis factors, transport proteins, homing receptors, and addressins.

14. The formulation of claim 13, wherein the polypeptide is selected from the group consisting of rennin; human growth hormone; bovine growth hormone; growth hormone releasing factor; parathyroid hormone; thyroid stimulating hormone; lipoproteins; α-1-antitrypsin; insulin; proinsulin; follicle stimulating hormone; calcitonin; luteinizing hormone; glucagon; a clotting factor such as factor VIIIC, factor IX, tissue factor, and von Willebrands factor; anti-clotting factors; atrial natriuretic factor; lung surfactant; a plasminogen activator; bombesin; thrombin; hemopoietic growth factor; tumor necrosis factor-α; tumor necrosis factor-β; enkephalinase; RANTES (regulated on activation normally T-cell expressed and secreted); human macrophage inflammatory protein (MIP-1-α); a serum albumin; mullerian-inhibiting substance; relaxin A-chain; relaxin B-chain; prorelaxin; gonadotropin-associated peptide; a microbial protein; DNase; inhibin; activin; vascular endothelial growth factor (VEGF); receptors for hormones or growth factors; integrin; protein A; protein D; rheumatoid factors; a neurotrophic factor; platelet-derived growth factor (PDGF); a fibroblast growth factor; epidermal growth factor (EGF); transforming growth factors (TGF); insulin-like growth factor-I; insulin-like growth factor-II; des(1-3)-IGF-I (brain IGF-I); insulin-like growth factor binding proteins; CD proteins; erythropoietin; osteoinductive factors; immunotoxins;; an interferon; colony stimulating factors (CSFs); interleukins (ILs); superoxide dismutase; T-cell receptors; surface membrane proteins; decay accelerating factor; antigens; transport proteins; homing receptors; addressins; regulatory proteins; immunoglobulin-like proteins; antibodies; and nucleases, or fragments thereof.

15. The formulation of claim 1, wherein biologically active molecule is selected from the group consisting of a lipid and a sterol.

16. The formulation of claim 1, wherein biologically active molecule is a small organic compound.

17. The formulation of any of claims 1-3, which is a precipitate.

18. The formulation of any of claims 1-3, which is a lyophilate.

19. A formulation comprising a precipitate or lyophilate of a polypeptide, which precipitate or lyophilate includes at least 50 percent (molar) polar protic organic solvent(s), and which formulation, when administered to a patient, releases said polypeptide at a rate providing an average steady state dosage of at least the ED₅₀ for the polypeptide for a period of at least 7 days.

20. A medicament for administeration to an animal, comprising the formulation of any of claims 1-3.

21. The medicament of claim 20, for administeration to a mammal.

22. The medicament of claim 20, for administeration to a human.

23. A method for manufacturing a medicament comprising formulating the formulation of any of claims 1-3 with a pharmaceutically acceptable excipient.

24. A method method for manufacturing a slow release formulation of a biologically active molecule, comprising (a) exposing said biologically active molecules to an organic solvent, and (b) forming a precipitate, lyophilate or crystal.

25. A method for conducting a pharmaceutical business comprising:
(a) preparing a formulation of any of claims 1-3;
(b) providing marketing and/or product literature for instructing healthcare providers on the use of said formulations; and
(c) providing a distribution network for deliverying said formuation to healthcare providers.
